(19) United States
(12) Patent Application Publication
(10) Pub. No.: US 2023/0281327 A1
(43) Pub. Date: Sep. 7, 2023

(54) UTILIZING A SWITCHBOARD MANAGEMENT SYSTEM TO EFFICIENTLY AND ACCURATELY MANAGE SENSITIVE DIGITAL DATA ACROSS MULTIPLE COMPUTER APPLICATIONS, SYSTEMS, AND DATA REPOSITORIES

(71) Applicant: Soojo, Inc., Danville, CA (US)

(72) Inventor: Joel Crockett, Danville, CA (US)

(21) Appl. No.: 18/175,811

(22) Filed: Feb. 28, 2023

Related U.S. Application Data
(60) Provisional application No. 63/268,838, filed on Mar. 3, 2022.

(51) Int. Cl. G06F 21/60

(52) U.S. Cl. CPC G06F 21/606

(57) ABSTRACT

The present disclosure relates to systems, non-transitory computer-readable media, and methods that utilize a switchboard management system as an integration framework for efficiently communicating and accurately populating digital fields between multiple applications while preserving the security and accuracy of sensitive information corresponding to various applications and data sources. For example, the disclosed systems assimilate a system of record and multiple supporting applications for launching via a single user platform. In particular embodiments, the disclosed systems generate digital mappings that map data fields (e.g., source fields and destination fields) between a system of record and a given supporting application and then automatically sync ground truth data between the applications. The disclosed system can also utilize machine learning approaches to determine inaccurate digital mappings, audit activity codes, and generate predicted workflows to further improve efficiency, flexibility and accuracy of implementing devices.

### CROSS-REFERENCE TO RELATED APPLICATIONS

This application claims priority to U.S. Provisional Application No. 63/268,838, entitled “UTILIZING A SWITCHBOARD MANAGEMENT SYSTEM TO EFFICIENTLY AND ACCURATELY MANAGE SENSITIVE DIGITAL DATA ACROSS MULTIPLE COMPUTER APPLICATIONS, SYSTEMS, AND DATA REPOSITORIES,” filed Mar. 3, 2022, the full disclosure of which is incorporated herein by reference.

### BACKGROUND

Recent years have seen significant technological development in computer networking systems, including digital communication processes, data transfer and storage, and data security measures. Indeed, many organizations have developed and/or currently utilize sophisticated software applications and data platforms specific to organizational requirements (such as medical software applications to implement disparate management functions across various clinical environments). In many cases, these organizations implement multiple, siloed computer systems that are individually designed to communicate with particular device hardware, perform certain technical functions, and provide inter-organization communications. For example, to accommodate interoperability between siloed systems (e.g., between a system of record and supporting applications), some conventional interfacing systems provide an interfacing platform. Unfortunately, a number of problems exist with conventional interfacing systems that lead to increased network inflexibility, data inaccuracies, and inefficient operation of implementing computing devices.

For example, some conventional interfacing systems require extensive data integration (e.g., via custom designed application programming interfaces) in order to facilitate back-end communication or data transfer between applications. Therefore, in many cases, such conventional interfacing systems are difficult to set up or build (e.g., in terms of computer programming and/or hardware integration) to achieve the custom connection. This is particularly true on larger scales for organizations where conventional interfacing systems are computationally expensive to maintain. In addition, some conventional interfacing systems are not compatible (or at least suffer from decreased performance) when interfacing between web-based applications and other types of applications. As a result, the over-rigidity of conventional interfacing systems largely prohibits adoption of new or upgraded software solutions and features as emerging technologies arise.

In addition to over-rigidity, some conventional interfacing systems inefficiently transmit data. For example, certain conventional interfacing systems require a multi-step data conversion (or data translation) process. To illustrate, some conventional interfacing systems transmit data between a first application and a second application by converting data in a first application language, format, etc. to a standardized messaging format (e.g., HL7 or Health Level 7). Then, the conventional interfacing systems convert the transmitted data from the standardized messaging format to a second application language, format, etc. This multi-step data conversion process can slow data transfers, decrease system bandwidth, and in some cases introduce conversion artifacts (e.g., errors, omissions, etc.).

Some conventional systems (with or without interfacing systems) also suffer from inefficient or complex user interface operations. For example, conventional systems process an inordinate amount of user interfaces and user interactions to transmit digital data. To illustrate, typical user interface operations of conventional systems involve excessive navigational steps to proceed back and forth between applications. In these scenarios for example, conventional systems process an excessive amount of interface commands to copy, paste, switch between applications, and access different data records. Exacerbating these issues, multiple applications and multiple data records can complicate and lengthen sequences of user interactions. In addition, data privacy and data security requirements can complicate the foregoing issues. For instance, sensitive information is often restricted with regards to communication between multiple applications or computing systems. Accordingly, utilizing conventional systems can lead to slower (or less secure) data transmission and/or increased processing errors.

### BRIEF SUMMARY

Embodiments of the present disclosure provide benefits and/or solve one or more of the foregoing or other problems in the art with systems, non-transitory computer-readable media, and methods that utilize a switchboard management system as an integration framework for efficiently communicating and accurately populating digital data fields between multiple applications while preserving the security and accuracy of sensitive information corresponding to various applications and data sources. To illustrate, the disclosed systems provide a digital platform that assimilates a system of record and multiple supporting applications for execution and management via a single user interface environment (e.g., a tabbed browser-like interface). In particular embodiments, the disclosed systems generate digital mappings that map data fields (e.g., source fields and destination fields) between a system of record and a given supporting application. After generating the digital mapping, the disclosed systems can automatically sync data (e.g., ground truth data) between the system of record and the given supporting application. For instance, based on accessing a data record in the system of record, the disclosed systems auto-populate data entries in a smart, secure clipboard and transfer to mapped fields in a launched supporting application.

In some embodiments, the disclosed systems utilize machine-learning models to provide additional or alternative features. For example, in certain implementations, the disclosed systems audit digital mappings by comparing digital mappings based on user selections with predicted digital mappings. Similarly, for instance, the disclosed systems can audit selected activity codes by comparing predicted activity codes from a machine-learning model. In turn, the disclosed systems can provide a notification (e.g., a warning message, error alert, etc.) regarding a digital mapping or a selected combination of activity codes. Still further, in some embodiments, the disclosed systems can track repetitive processes (e.g., utilizing a machine-learning model) and generate predicted workflows for automation.

Further, some embodiments of the disclosed systems implement a third-party interchange system for interactively exchanging data between third-party servers (e.g., care coordination data). For example, the disclosed systems can dynamically exchange digital referral documents, share digital files (e.g., images, patient charts, customer data, tasks, appointments, etc.), and provide customizable status updates with a third-party server. In some cases, the disclosed systems provide interactive options when exchanging data with a third-party server (e.g., for commenting on digital tasks, accepting digital tasks, or responding with queries). In certain implementations, the disclosed systems utilize access permissions to control how third-party servers can access, edit, or share exchanged data.

Additional features and advantages of one or more embodiments of the present disclosure are outlined in the following description.

### BRIEF DESCRIPTION OF THE DRAWINGS

The detailed description explains one or more embodiments with additional specificity and detail through the use of the accompanying drawings, as briefly described below.

FIG. 1 illustrates a computing system environment for implementing a switchboard management system in accordance with one or more embodiments.

FIG. 2 illustrates a switchboard management system managing digital information between client devices and corresponding applications in accordance with one or more embodiments.

FIG. 3 illustrates a switchboard management system using a digital mapping to modify a plurality of destination fields in accordance with one or more embodiments.

FIGS. 4A-4K illustrate a switchboard management system providing user interfaces on a computing device in accordance with one or more embodiments.

FIGS. 5A-5B illustrate a switchboard management system training and implementing an activity code prediction model in accordance with one or more embodiments.

FIGS. 6A-6B illustrate a switchboard management system training and implementing a digital mapping prediction model in accordance with one or more embodiments.

FIG. 7 illustrates a client device implementing a third-party interchange system in accordance with one or more embodiments.

FIG. 8 illustrates a switchboard management system generating a predicted digital workflow for automating in accordance with one or more embodiments.

FIG. 9 illustrates an example schematic diagram of a switchboard management system in accordance with one or more embodiments.

FIG. 10 illustrates a flowchart of a series of acts for modifying a plurality of destination fields in accordance with one or more embodiments.

FIG. 11 illustrates a block diagram of an example computing device for implementing one or more embodiments of the present disclosure.

### DETAILED DESCRIPTION

One or more embodiments described herein include a switchboard management system that dynamically integrates a primary application (e.g., a system of record) and a variety of additional applications (e.g., supporting applications) within a unified platform. In particular, the switchboard management system can intelligently map data fields in the primary application to data fields in an additional application to efficiently and accurately populate digital data fields across applications while preserving security of isolated digital data silos. In this manner, the switchboard management system can effectively provide a flexible unifying platform that allows for rapid, user driven integration of software applications.

As mentioned above, in some embodiments, the switchboard management system provides a unified platform in which a user can select a primary application and one or more additional applications (e.g., supporting applications) to launch, access, and exchange digital data. Via a unified user interface window, the switchboard management system can execute acts or algorithms to more efficiently, accurately, and flexibly communicate digital data (e.g., electronic health record data) between the primary application and the additional applications. For example, the switchboard management system can automatically extract ground truth digital data entries from a data record accessed via the primary application. In turn, the switchboard management system can automatically transfer the extracted ground truth digital data entries into corresponding fields (e.g., destination fields) within an additional application. For instance, the switchboard management system transfers the extracted ground truth digital data entries in response to a client device accessing (e.g., selecting a tab that corresponds to) the additional application within the unified user interface window.

In one or more embodiments, the switchboard management system utilizes a digital mapping to link a primary application and an additional application in order to provide ground truth digital data entries between applications. In particular embodiments, a digital mapping assigns source fields in the primary application to corresponding destination fields in an additional application. In certain implementations, the switchboard management system generates the digital mapping in response to user inputs indicating the source fields in the primary application and user inputs indicating the destination fields in the additional application. For example, the switchboard management system generates mapping pairs based on the user inputs such that the digital mapping is composed of a set of mapping pairs (e.g., a first source field corresponding to a first destination field, a second source field corresponding to a second destination field, and so forth). In certain embodiments, the digital mapping is also editable (e.g., such that a user can add, remove, or revise mapping pairs).

After the switchboard management system generates the digital mapping for the primary application and the additional application, no further remapping between these applications is needed. For example, the switchboard management system associates the digital mapping with a client device (or client application account). Moreover, the switchboard management system can apply the digital mapping in the future when the client device launches the primary application and the additional application within the unified user interface window.

In one or more embodiments, the switchboard management system utilizes a digital clipboard to transfer ground truth digital data entries between source fields in the primary application and destination fields in the additional application. For example, the switchboard management system can populate the digital clipboard with ground truth digital data entries that correspond to mapped source fields based on accessing a data record via the primary application. The switchboard management system can then transfer the ground truth digital data entries in the digital clipboard to destination fields in accordance with the digital mapping. For instance, in response to a user switching active windows to the additional application, the switchboard management system can automatically transfer the ground truth digital data entries into the destination fields.

In certain implementations, the switchboard management system utilizes a digital clipboard with increased security and/or privacy measures to protect and preserve sensitive digital data. For example, the switchboard management system can clear the digital clipboard at select times to maintain data security and prevent incidental/incorrect data transfers. To illustrate, the switchboard management system can clear the digital clipboard of the ground truth digital data entries corresponding to a first data record in response to detecting the client device navigating away from the first data record in the primary application. For instance, the switchboard management system deletes the ground truth digital data entries from the digital clipboard in response to receiving an indication to exit or log out of the primary application, close the first data record, or navigate to a landing/home page within the primary application. As another example, the switchboard management system can clear the digital clipboard in response to accessing a second data record via the primary application. In this example, the switchboard management system can subsequently repopulate the digital clipboard with different ground truth digital data entries that correspond to the second data record.

In addition to dynamically managing the digital clipboard, certain embodiments of the switchboard management system also clear transferred data in additional applications in response to interactions or modifications within the primary application. For example, if a primary application accesses a new digital record, the switchboard management system can clear the digital clipboard and also clear transferred data in an additional application. In this manner, the switchboard management system can ensure that launched applications are synced to a particular data record in the primary application (thereby avoiding cross-transfer of secure/private data).

As mentioned above, the switchboard management system can also provide a variety of additional features. For example, in certain implementations, the switchboard management system dynamically links multiple client devices implementing the switchboard management system. To illustrate, the switchboard management system can provide secure digital communications between client devices (and corresponding client applications). Additionally or alternatively, the switchboard management system can provide secure digital communications between devices implementing the switchboard management system and third-party devices. Such secure communications may include correspondence via email, instant messaging or chat, text (SMS) messaging, application messaging, system alerts, shared calendars (or other shared files), fax, voice calling, video calling, etc.

In some embodiments, the switchboard management system monitors and tracks digital activity data for client devices implementing the switchboard management system. For example, in certain implementations, the switchboard management system audits digital mappings generated based on user inputs to select source fields and destination fields. To illustrate, the switchboard management system can generate a predicted digital mapping utilizing a machine-learning model trained on digital mappings for a particular client device or multiple client devices (e.g., for an organization or similar user accounts). Using the predicted digital mapping, the switchboard management system can determine whether the actual digital mapping includes a mapping anomaly (e.g., a potential error or discrepancy). In turn, the switchboard management system can notify the client device and/or an administrator device regarding the mapping anomaly.

As another example, the switchboard management system can monitor activity codes to audit and/or recommend activity codes. For example, the switchboard management system can similarly generate predicted activity codes utilizing a machine-learning model trained to select an optimal combination of activity codes (e.g., according to predefined optimization metrics). Accordingly, the switchboard management system can provide a set of features to the machine-learning model for automatically generating a recommended combination of activity codes. In certain embodiments, the switchboard management system compares the recommended combination of activity codes and a selected combination of activity codes to determine whether the selected combination of activity codes is accurate or optimal (e.g., according to one or more threshold values). If not accurate or optimal, the switchboard management system can notify the client device (and/or an admin device) regarding the selected activity codes and the recommended combination of activity codes.

In yet another example, the switchboard management system can monitor and track user activity data for one or more client devices. Based on the monitored user activity data, the switchboard management system can generate a predicted workflow utilizing a machine-learning model trained to cluster, classify, or segment digital user activities into predicted digital workflows. In turn, one or more embodiments of the switchboard management system automate the predicted digital workflow (e.g., by creating one or more user interface elements that, when interacted with by the client device, perform the predicted digital workflow).

In some embodiments, the switchboard management system implements a third-party interchange system for linking client devices of the switchboard management system with third-party servers. In particular, implementing computing devices of the switchboard management system can interactively exchange data between third-party servers (e.g., care coordination data) by using the third-party interchange system. For example, the third-party interchange system can transmit (and receive) myriad types of data, such as recommended referrals, follow-up services, instructions, assignments, permissions, documentation, files, test results, images, records, medical history, receipts, reports, prescriptions, etc.

As mentioned above, a number of problems exist with conventional interfacing systems, particularly with regard to increased network inflexibility, inefficient data transfer methods, and data processing errors.

In contrast to the foregoing problems, the switchboard management system can provide several improvements over conventional systems. For example, the switchboard management system can provide increased network flexibility. In particular, the switchboard management system can intuitively and quickly link and manage different applications (e.g., a primary application and an additional application in seconds). Indeed, unlike some conventional interfacing systems, the switchboard management system does not require custom application programming interfaces or extensive data integration. Instead, the switchboard management system can flexibly generate a digital mapping for any two applications based on user inputs indicating source fields and destination fields within the applications. In this manner, the switchboard management system can easily integrate myriad different application types together, which provides increased system flexibility to change existing integrated applications or adopt different ones.

In addition to increased system flexibility, the switchboard management system can provide additional data-transmission efficiencies. For example, the switchboard management system can utilize a digital clipboard to transfer ground truth digital data entries from a primary application to an additional application based on a digital mapping. By using this approach and eliminating the processing steps for data conversion, the switchboard management system can decrease bandwidth consumption and/or decrease a processing time for data transmission between applications.

The switchboard management system can also provide improved, more efficient user interfaces relative to conventional systems. For example, unlike some conventional systems that require excessive navigational inputs and other interface commands, the switchboard management system provides an intuitive user interface that guides user selections for specific source fields and destination fields within applications. From the user selections, the switchboard management system can generate a digital mapping that, from then on, automatically applies to user interactions between the mapped applications. This approach can greatly reduce the amount of user interactions that the switchboard management system processes, which leads to faster data transmissions and/or decreased processing errors.

In addition, the switchboard management system can provide more secure communication between a primary application and an additional application. In particular, the switchboard management system is compliant with today’s stringent data security and data privacy requirements. For example, by utilizing a smart, secure digital clipboard, the switchboard management system can detect a user interaction to navigate away from a data record in the primary application. In turn, the switchboard management system can delete ground truth digital data entries from the digital clipboard. Additionally or alternatively, in some embodiments, the switchboard management system can also delete the ground truth digital data entries from an additional application such that the destination fields are cleared. In this manner, the switchboard management system can avoid incidental/incorrect data transfers between applications.

Further, the switchboard management system can provide one or more improvements through various other features. For example, the switchboard management system can provide an activity code prediction model that improves an accuracy of selected activity codes based on one or more of a wide variety of digital inputs (e.g., to more accurately and efficiently track certain activities). Similarly, the switchboard management system can provide a digital mapping prediction model that improves the accuracy of digital mappings (e.g., by identifying certain mapping anomalies and/or recommending a specific digital mapping). In addition, the switchboard management system can provide a third-party interchange system that improves real-time communication and efficiency of data transfer with third-party server(s). Still further, the switchboard management system 104 can provide a workflow prediction model that improves accuracy of digital recommendations (e.g., a recommended digital workflow) that accounts for user-specific digital actions and efficiently implements new workflows.

As illustrated by the foregoing discussion, the present disclosure utilizes a variety of terms to describe features and benefits of the switchboard management system. Additional detail is now provided regarding the meaning of these terms. For example, as used herein, the term “application” refers to a software program executable on a computing device. For example, an application can include web-based applications, stand-alone applications, web browsers, email programs, word processors, utilities, etc.

Relatedly, the terms “primary application” and “additional application” refer to designations or classes of an application. In particular embodiments, a primary application includes a main application, a system of record, or a core system. In contrast, an additional application includes a separate application. For example, additional applications can include secondary or supporting applications to be used with a primary application. Moreover, a primary application and an additional application can correspond to myriad different fields of use (e.g., healthcare) without limitation. Indeed, a primary application and an additional application can correspond to one or more of siloed data storage systems, disparate data platforms, different communication systems, customer/client-specific systems, etc.

In addition, as used herein, the term “field” refers to a digital element, region, or unit for storing digital data entries. For example, a field can include a cell, row, column, box, or line (e.g., in a database, array, storage repository, or user interface). A field can include a variety of user interface elements, such as a text box that portrays digital information via a display screen. As additional examples, a field can include the spatial regions that correspond to a label, category, tab, or folder.

Relatedly, the terms “source field” and “destination field” refer to designations or classes of fields. For example, a source field includes a field from a primary application. Similarly, for instance, a destination field can include to a field from an additional application.

As further used herein, the term “ground truth digital data entry” refers to a digital information corresponding to a field. In particular embodiments, a ground truth digital data entry refers to a value corresponding to a source field of a primary application. For instance, a ground truth digital data entry may include “Jan. 1, 2000” for a source field of “Date of Birth.”

In addition, as used herein, the term “digital mapping” refers to a data structure for aligning fields. In particular embodiments, a digital mapping can include a data structure for storing mapping pairs (e.g., field assignments that each relate a source field to a destination field and/or a destination field to a source field). For example, a digital mapping may include mapping pairs arranged in an index, vector, table, nodal graph, data tree, etc.

As also used herein, the term “digital clipboard” refers to a data structure for short-term storage of application data. In particular embodiments, a digital clipboard can include ground truth digital data entries that are copied from source fields (e.g., for transfer from the digital clipboard to destination fields according to a digital mapping). In certain implementations, the switchboard management system can automatically copy, transfer, and clear ground truth digital data entries in a digital clipboard in response to user interactions with a primary application and/or an additional application.

As used herein, the term “data record” refers to digital information corresponding to a particular user, account, customer, client, patient, and/or organization. Examples of data records include electronic health records, customer accounts, client files, user profiles, etc.

As further used herein, the term “prompt” refers to a user interface element. In particular embodiments, a prompt can include a user interface element that guides or requests user interaction. For example, a prompt may include a request, within a user interface, to indicate a source field or a destination field (e.g., via clicks, haptic inputs, etc.).

In addition, as used herein, the term “machine-learning model” refers to a computer model or computer representation that can be tuned (e.g., trained) based on inputs to approximate unknown functions. For example, a machine-learning model may include one or more of a decision tree (e.g., a gradient boosted decision tree), a linear regression model, a logistic regression model, association rule learning, inductive logic programming, support vector learning, a Bayesian network, a regression-based model, principal component analysis, a neural network (e.g., convolutional neural network or recurrent neural network), or a combination thereof.

Accordingly, the term “activity code prediction model” refers to a machine-learning model for determining predicted activity codes. In particular embodiments, an activity code prediction model can include one or more machine-learning models that generate predicted activity codes based on one or more features corresponding to an account, user, or treatment

Further, the term “digital mapping prediction model” refers to a machine-learning model for determining a predicted digital mapping between fields. In particular embodiments, a digital mapping prediction model can include one or more machine-learning models that generate a predicted digital mapping based on field features.

In addition, the term “workflow prediction model” refers to a machine-learning model for determining a predicted digital workflow. In particular embodiments, a workflow prediction model can include one or more machine-learning models that generate a predicted digital workflow based on user activity data (e.g., individual digital actions, sequences of digital actions, repeated digital actions, etc.).

As also used herein, the term “mapping anomaly” refers to a discrepancy or potential error of a digital mapping. In particular embodiments, a mapping anomaly refers to dissimilar digital mappings, unusual digital mappings, etc. in relation to other digital mappings (e.g., for a same user, a different user, or multiple users). For example, a mapping anomaly can include a mapping pair that comprises a “Date of Birth” field in a primary application and a “Last Name” field in an additional application.

Relatedly, as used herein, the term “digital workflow” refers to a plurality of related user activities performed by a computing device associated with a user. In particular, a digital workflow can refer to a subset of digital activities that are frequently performed together. For instance, a digital workflow can refer to a subset of digital activities frequently performed within a user session. In particular embodiments, the subset of digital activities comprising a digital workflow are order agnostic and may, for instance, be an identifiable (e.g., numbered) collection of frequently co-occurring user activities. For example, a digital workflow can include performance of a common series or collection of user activities, such as a subset of digital actions for analyzing digital data, updating electronic health records, and generating graphic visualizations.

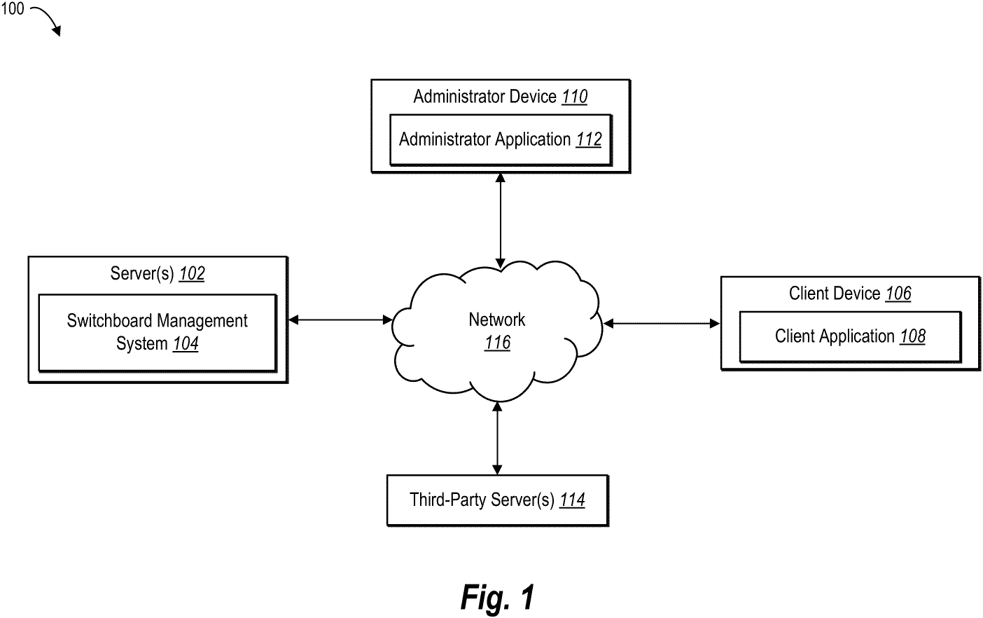

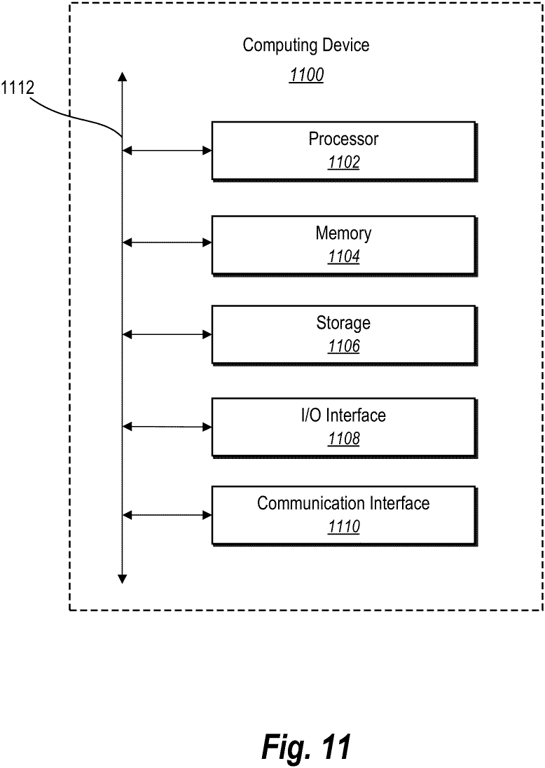

Additional detail will now be provided regarding the switchboard management system in relation to illustrative figures portraying example embodiments and implementations of the switchboard management system. For example, FIG. 1 illustrates a computing system environment (or “environment”) 100 for implementing a switchboard management system 104 in accordance with one or more embodiments. As shown in FIG. 1, the environment 100 includes server(s) 102, a client device 106, an administrator device 110, third-party server(s) 114, and a network 116. Each of the components of the environment 100 communicate (or are at least configured to communicate) via the network 116, and the network 116 may be any suitable network over which computing devices can communicate. Example networks are discussed in more detail below in relation to FIG. 11.

As further illustrated in FIG. 1, the environment 100 includes the server(s) 102. In some embodiments, the server(s) 102 comprises a content server and/or a data collection server. Additionally or alternatively, the server(s) 102 comprise an application server, a communication server, a web-hosting server, a social networking server, or a digital content management server.

Moreover, as shown in FIG. 1, the server(s) 102 implement the switchboard management system 104. The switchboard management system 104 can efficiently modify destination fields in an application based on a digital mapping. To illustrate, in one or more embodiments, the switchboard management system 104 identifies a primary application and a plurality of additional applications (where the primary application includes a set of ground truth digital data entries for a data record). In certain embodiments, the switchboard management system 104 then detects, via a user interface, a first set of user inputs indicating a plurality of source fields corresponding to the set of ground truth digital data entries for the primary application. Additionally, in some embodiments, the switchboard management system 104 detects, via an additional user interface, a second set of user inputs indicating a plurality of destination fields of a first application of the plurality of additional applications. In turn, one or more embodiments of the switchboard management system 104 generate a digital mapping assigning the plurality of source fields of the primary application to the plurality of destination fields of the first application based on the first set of user inputs and the second set of user inputs. Moreover, in certain implementations, the switchboard management system 104 modifies the plurality of destination fields in the first application to include the first set of ground truth digital data entries for the first data record in accordance with the digital mapping.

As shown in FIG. 1, the environment 100 includes the client device 106. The client device 106 can include one of a variety of computing devices, including a smartphone, tablet, smart television, desktop computer, laptop computer, virtual reality device, augmented reality device, or other computing device as described in relation to FIG. 11. Although FIG. 1 illustrates a single client device 106, in some embodiments the environment 100 includes multiple client devices 106. In these or other embodiments, the client device 106 further communicates with the server(s) 102 via the network 116. For example, the client device 106 receives user input for indicating source fields and destination fields to the server(s) 102.

As shown, the client device 106 includes a client application 108. In particular embodiments, the client application 108 comprises a web application, a native application installed on the client device 106 (e.g., a mobile application, a desktop application, etc.), or a cloud-based application where part of the functionality is performed by the server(s) 102. In some embodiments, the client application 108 presents or displays information to a user associated with the client device 106, including unified user interface window for launching and accessing primary and additional applications.

In additional or alternative embodiments, the client application 108 (e.g., as a switchboard application) represents and/or provides the same or similar functionality as described herein in connection with the switchboard management system 104. In some implementations, the client application 108 supports the switchboard management system 104 on the server(s) 102. Indeed, in one or more embodiments, the client device 106 includes all, or a portion of, the switchboard management system 104.

Additionally shown in FIG. 1, the environment 100 includes the administrator device 110. Like the client device 106, the administrator device 110 can include one of a variety of computing devices, including a smartphone, tablet, smart television, desktop computer, laptop computer, virtual reality device, augmented reality device, or other computing device as described in relation to FIG. 11. In particular, the administrator device 110 includes an administrator application 112. Similar to the client application 108, the administrator application 112 can include a variety of different applications. Moreover, in some embodiments, the administrator application 112 presents or displays information to a user associated with the administrator device 110, including a user interface window for receiving alerts regarding digital mapping anomalies, recommended activity codes, or predicted digital workflows.

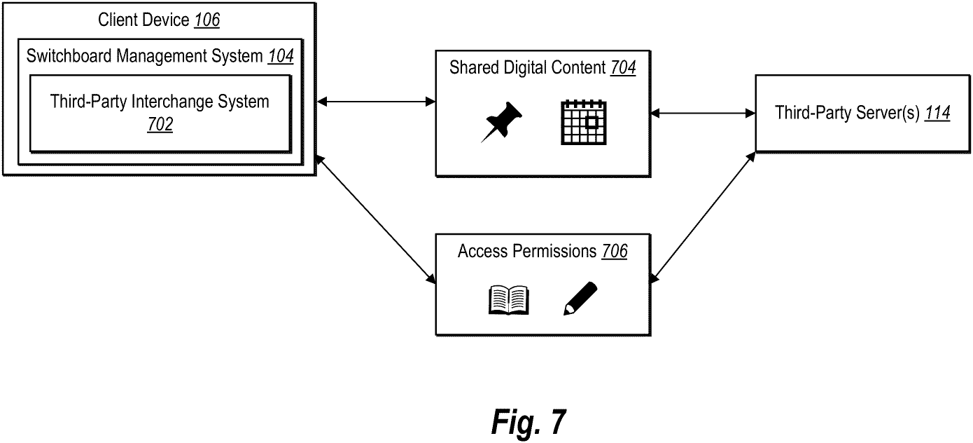

Further shown, the environment 100 includes the third-party server(s) 114. The third-party server(s) 114 can include one or more of a variety of servers as described above for the server(s) 102. In particular embodiments, the third-party server(s) 114 can correspond to one or more computing systems of other (e.g., third-party) organizations. In certain implementations, the third-party server(s) 114 implement the switchboard management system 104. In other implementations, the third-party server(s) 114 do not implement the switchboard management system 104. In either case, the switchboard management system 104 can utilize a third-party interchange system to exchange various data (e.g., care coordination data) as described further below in relation to FIG. 7.

In some embodiments, though not illustrated in FIG. 1, the environment 100 has a different arrangement of components and/or has a different number or set of components altogether. For example, in certain embodiments, the environment 100 does not include an administrator device and/or a third-party server. As another example, the environment 100 includes one or more storage servers or databases (e.g., hosted on third-party servers for storing data records and/or care coordination data). In yet another example, the client device 106 communicates directly with the server(s) 102, bypassing the network 116.

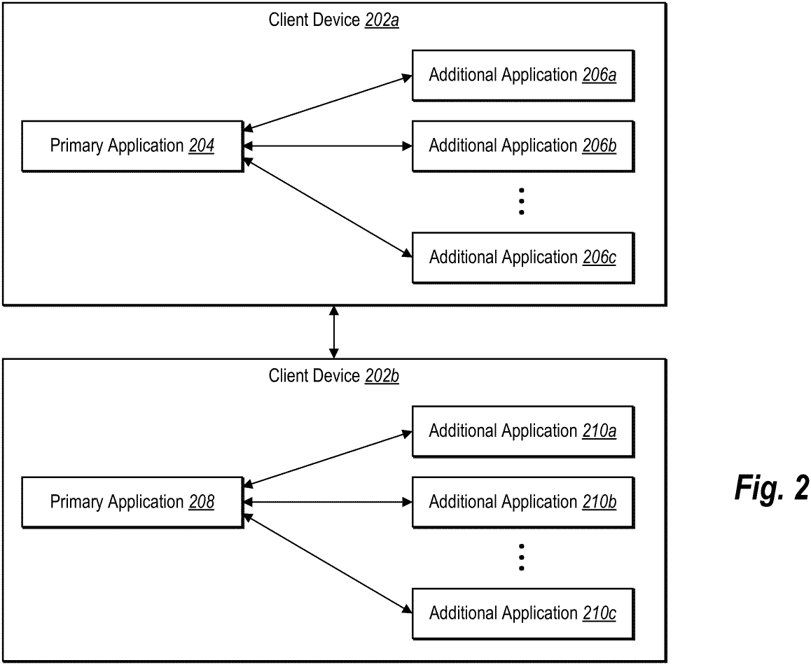

As mentioned above, the switchboard management system 104 can provide efficient communication between different applications and also between client devices. FIG. 2 illustrates the switchboard management system 104 linking client devices 202a-202b to each other and to corresponding applications in accordance with one or more embodiments. In some embodiments, the client devices 202a-202b correspond to a same organization, facility, department, etc. In other embodiments, the client devices 202a-202b correspond to different organizations, facilities, departments, etc. Therefore, linking the client devices 202a-202b allows the switchboard management system 104 to provide secure communication (e.g., internally or externally) between the client devices 202a-202b.

To illustrate, the switchboard management system 104 allows the client devices 202a-202b to access one or more accounts that are linked together via the switchboard management system 104. For example, the client device 202a can independently access a first account via a client application for the switchboard management system 104. Similarly, the client device 202b can independently access a second account via a client application for the switchboard management system 104.

After logging into the first and second accounts (e.g., via respective usernames and passwords), the client devices 202a-202b can securely communicate with each other via the switchboard management system 104. For example, in one or more embodiments, the switchboard management system 104 provides real-time communication between the client devices 202a-202b. To illustrate, the switchboard management system 104 can provide secure communication between the client devices 202a-202b via email, instant messaging or chat, text (SMS) messaging, application messaging, system alerts, shared calendars (or other shared files), fax, voice calling, video calling, etc. Moreover, it can be appreciated that such secure communication between the client devices 202a-202b may include transmission of myriad types of digital content, such as electronic health records, customer reports, research data, lab results, etc. Care coordination data and other examples of transmitted data are discussed below in relation to FIG. 7.

Further shown in FIG. 2, the switchboard management system 104 provides the client device 202a access to a primary application 204 and additional applications 206a-206c. Similarly, the switchboard management system 104 provides the client device 202b access to a primary application 208 and additional applications 210a-210c.

In particular embodiments, the switchboard management system 104 provides each of the client devices 202a-202b specialized access to these applications. For example, the switchboard management system 104 provides, for display on the client devices 202a-202b, respective unified user interfaces for launching and accessing multiple applications from a single, centralized location.

This specialized, central access is integral to the switchboard management system 104 because data privacy laws and data security measures typically require a data repository for a first application be kept separate, siloed, or otherwise independent of a data repository for a second application. Accordingly, in some circumstances, the first application cannot access the data repository of the second application, and the second application cannot access the data repository of the first application without this specialized, central access provided by the switchboard management system 104. That is, in these circumstances, without the switchboard management system 104, the first application can only access stored data within the data repository for the first application, and the second application can only access stored data within the data repository for the second application.

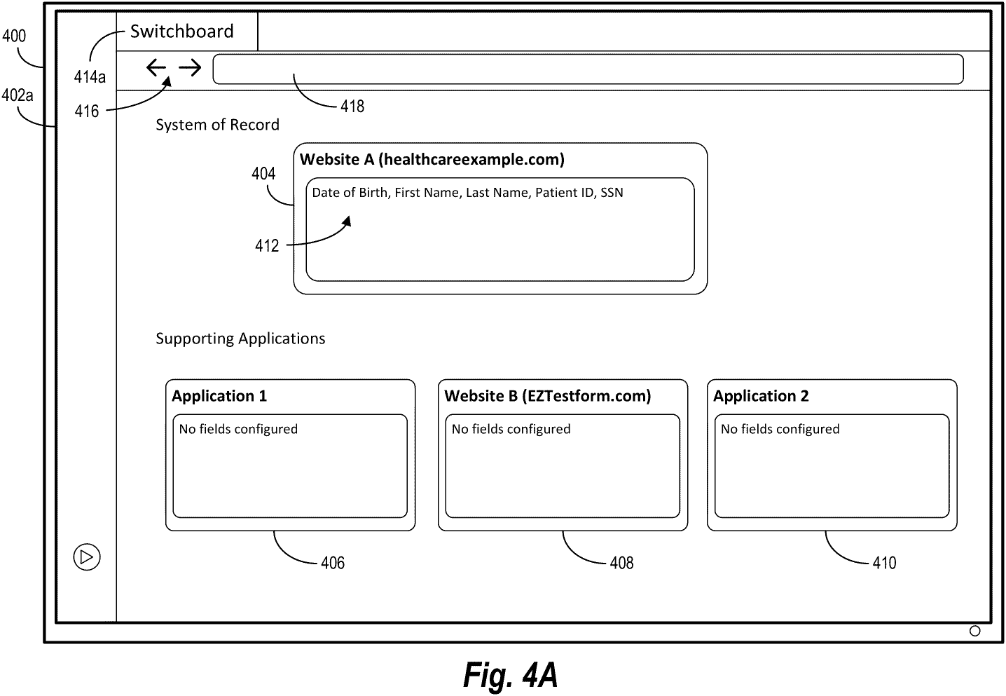

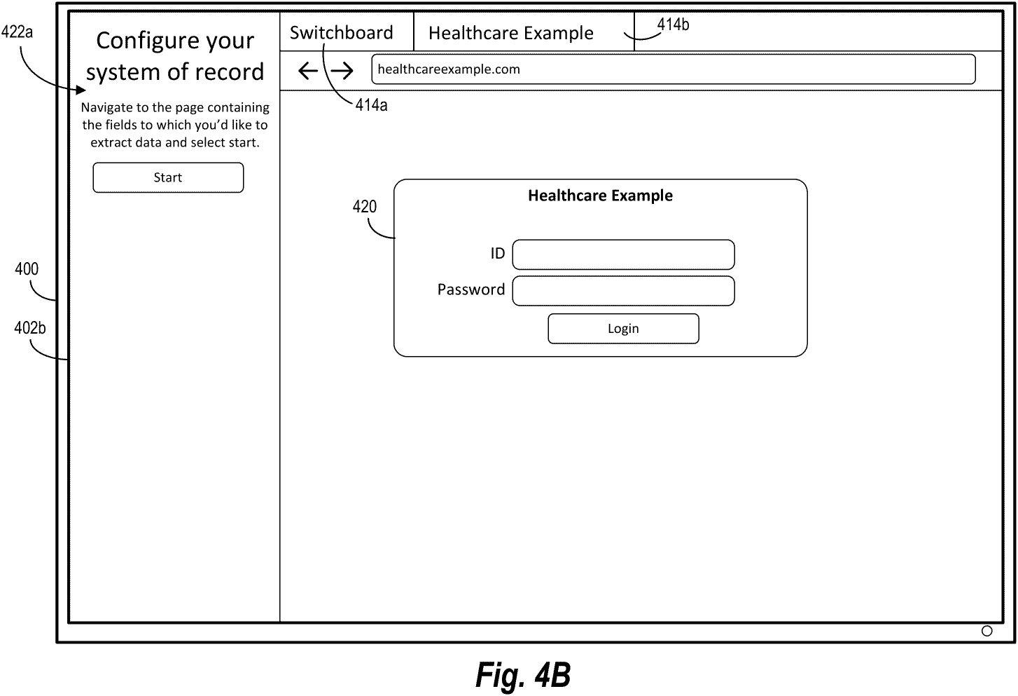

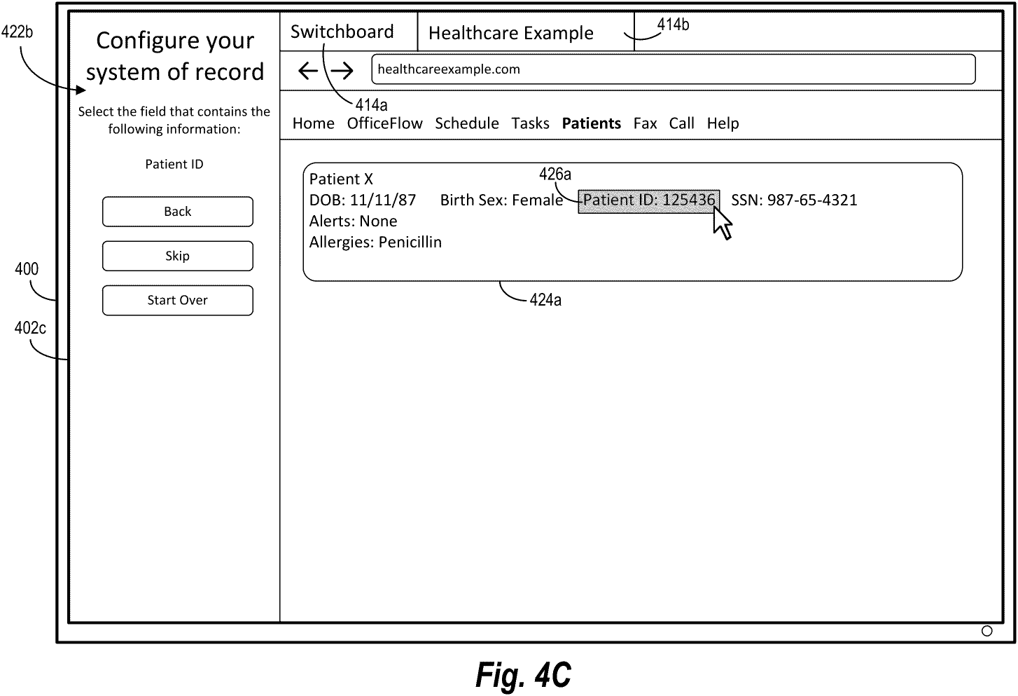

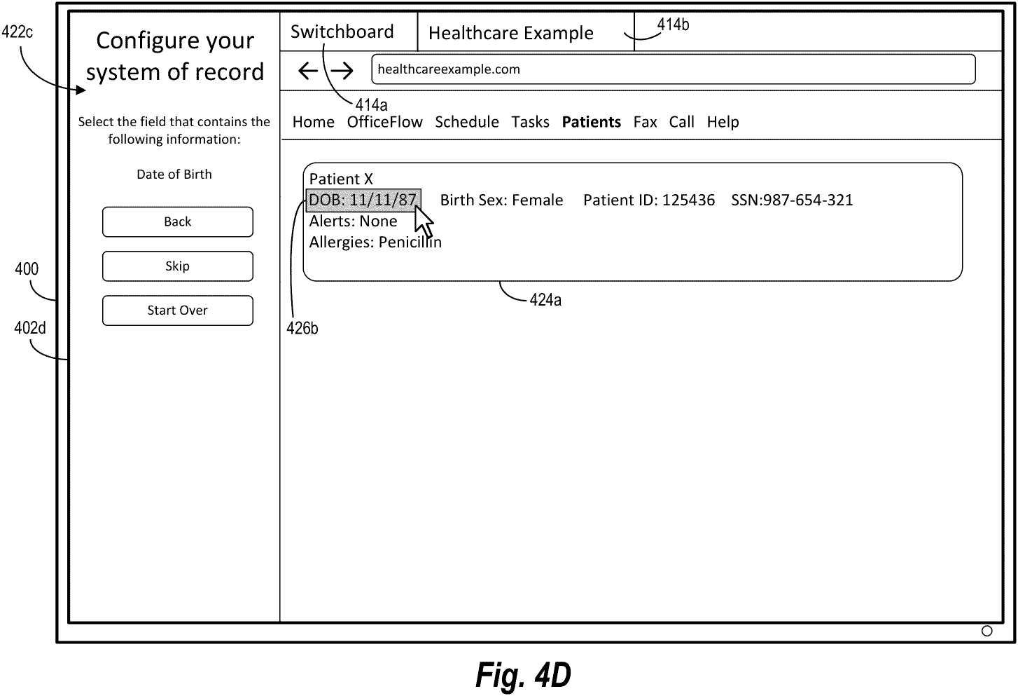

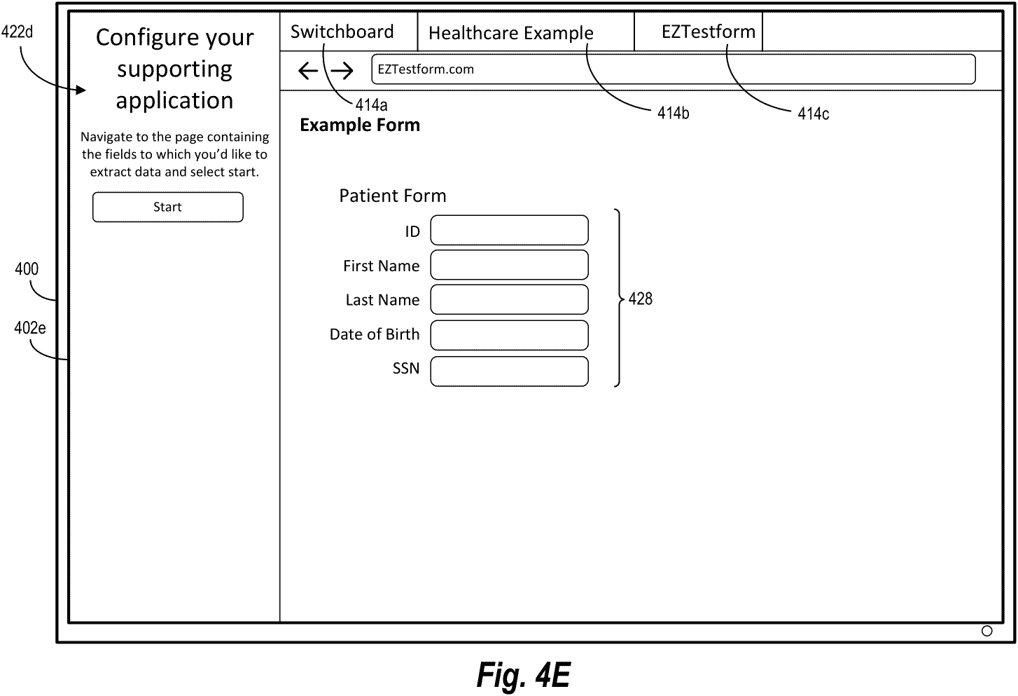

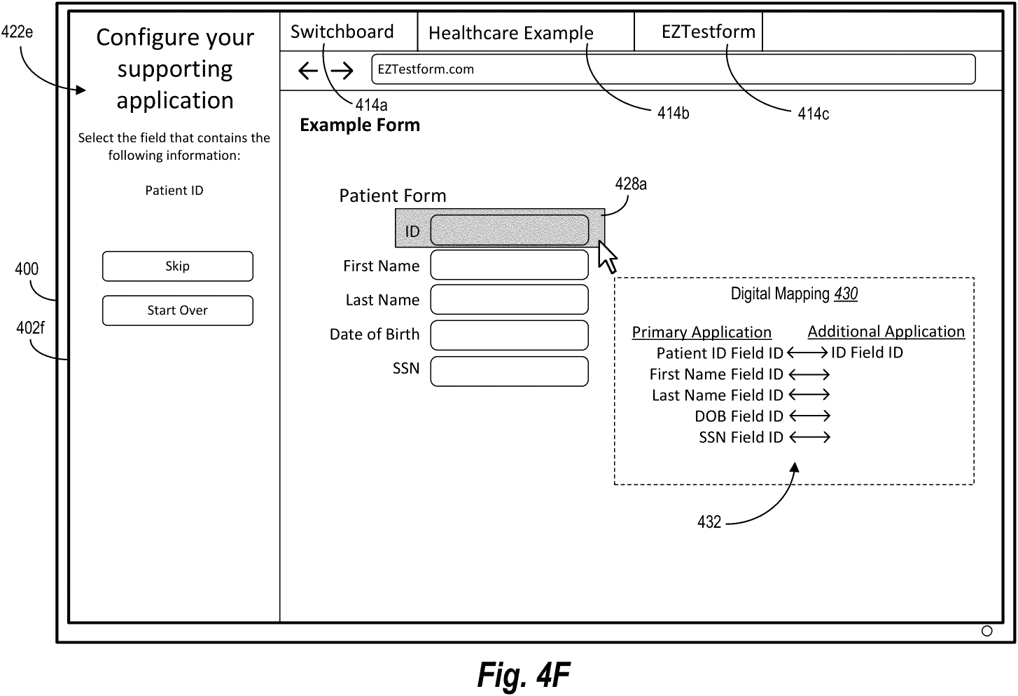

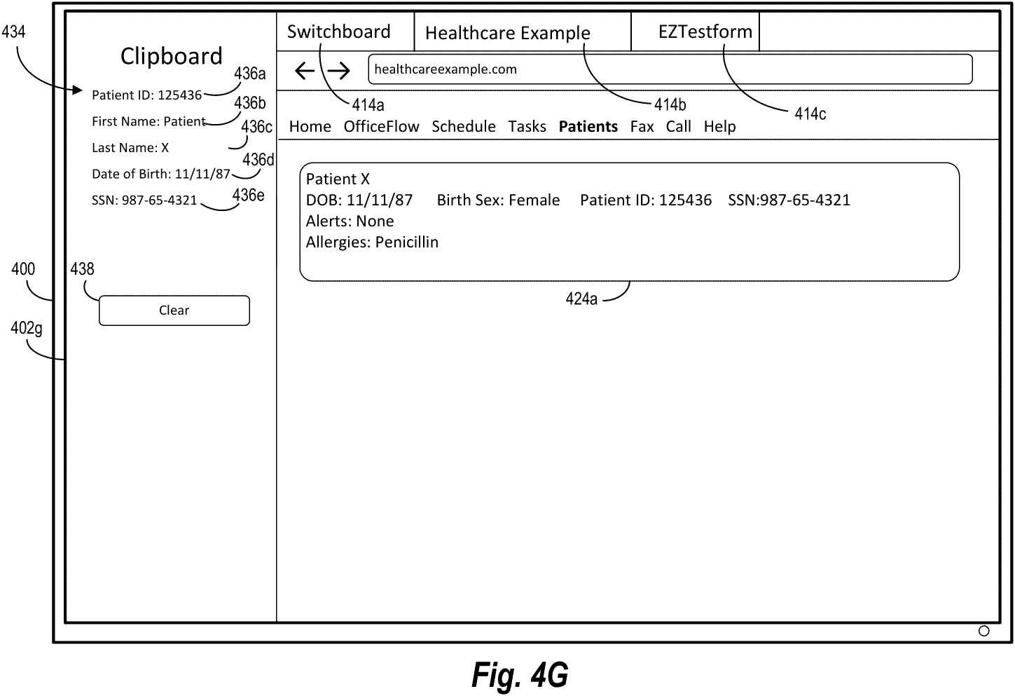

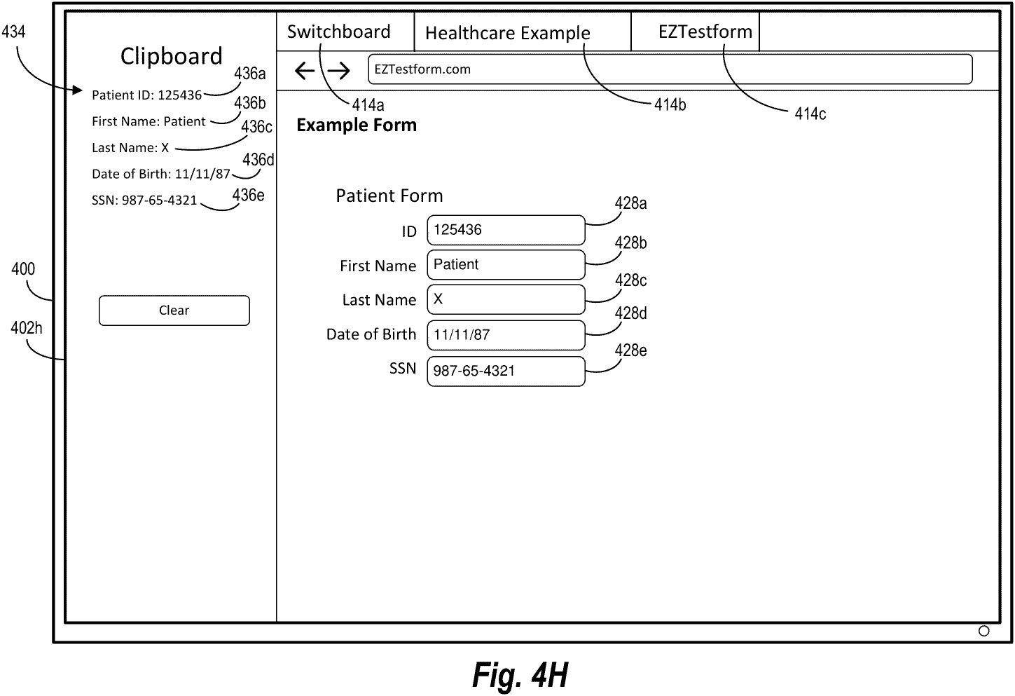

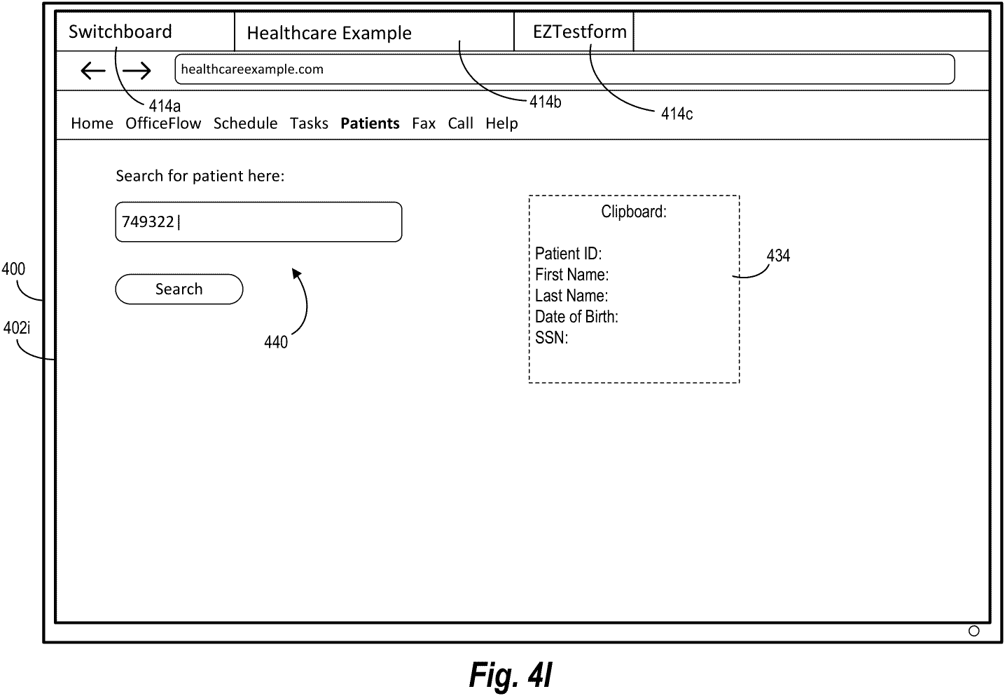

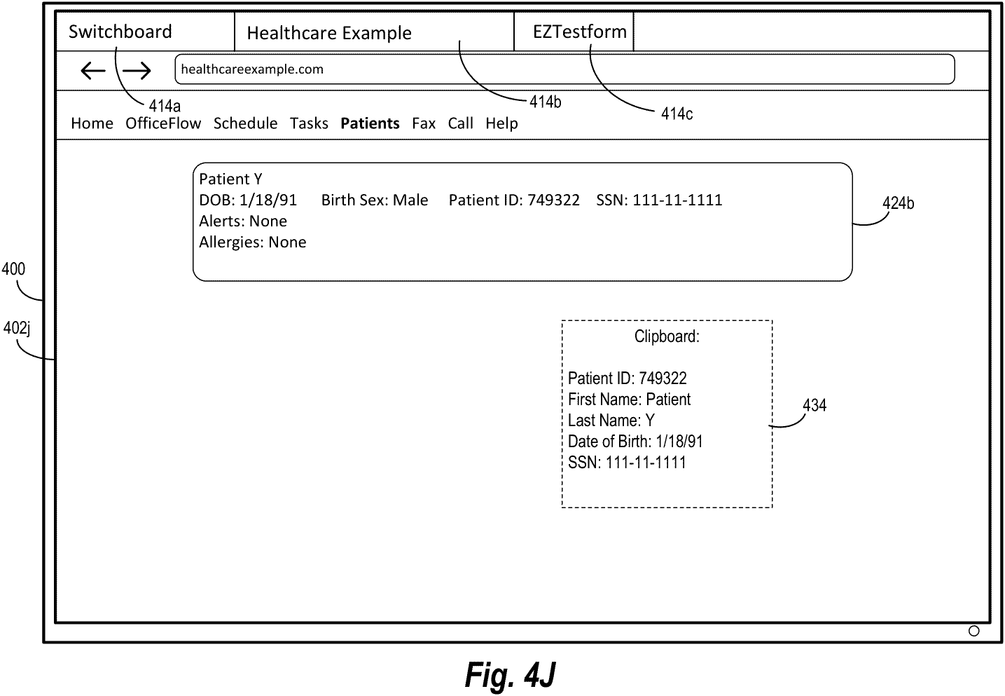

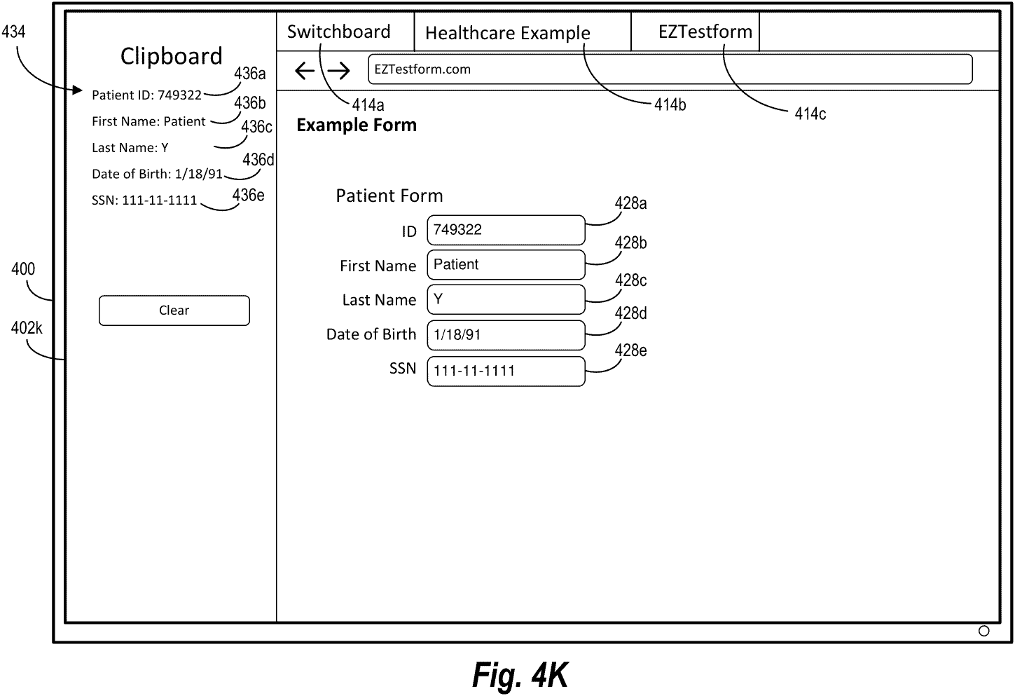

To illustrate this specialized, central access, the switchboard management system 104 can accept login credentials and grant the client device 202a access to a personalized switchboard account (e.g., via a client application). Within the account, the switchboard management system 104 can provide the client device 202a with a unified user interface displaying each of the primary application 204 and the additional applications 206a-206c (e.g., as shown in FIG. 4A). Via the unified user interface, the switchboard management system 104 can receive indications from the client device 202a to simultaneously launch (or open together) the primary application 204 and one or more of the additional applications 206a-206c. In this manner, the switchboard management system 104 can efficiently open multiple applications at a time.

In these or other embodiments, the primary application 204 and the primary application 208 correspond to systems of record. In certain implementations, the primary application 204 and the primary application 208 correspond to a same system of record, while in other implementations, different systems of record. Examples of the primary application 204 and the primary application 208 include applications for healthcare systems, communication systems, customer/client systems, data storage systems, etc.

Moreover, the primary application 204 and the primary application 208 include applications that comprise ground truth digital data entries. As will be explained in greater detail below, the switchboard management system 104 uses digital mappings to accurately and efficiently update additional applications with the ground truth digital data entries included in a primary application. Specifically, the primary application 204 comprises ground truth digital data entries that function as the data source for the additional applications 206a-206c. Similarly, the primary application 208 comprises ground truth digital data entries that function as the data source for the additional applications 210a-210c.

In one or more embodiments, the additional applications 206a-206c and the additional applications 210a-210c include applications that correspondingly relate to, support, or rely on the primary application 204 and the primary application 208. Further, the additional applications 206a-206c and the additional applications 210a-210c can similarly include a wide variety of applications for healthcare systems, communication systems, customer/client systems, data storage systems, etc.

In addition to primary applications and additional applications, it can be appreciated that the client devices 202a-202b also include access to other features and applications. For example, the switchboard management system 104 does not prohibit or impede standard browser function for web-based activities (e.g., entering universal resource locators, navigating to webpages, refreshing pages, standard copying and pasting, or interacting with forward/backward navigation buttons).

Although FIG. 2 only shows the client devices 202a-202b, in certain implementations, the switchboard management system 104 includes additional client devices in communication with each other. As one example, multiple client devices may be logged into a single account. In this example, the switchboard management system 104 can share secure communications between the client devices logged into the same account. For instance, the switchboard management system 104 may provide an instant message from the client device 202a to all client devices associated with a particular account, such as the client device 202b (e.g., a desktop computer) and an additional client device (e.g., a wearable device or tablet device).

Further, it can be appreciated that the switchboard management system 104 may provide the foregoing features (and others of the present disclosure) based on a software or coding platform of various kinds. In some embodiments, such software or coding platform facilitates custom development and creation of applications. Accordingly, in one or more embodiments, the switchboard management system 104 is partially based on a software-building framework, such as Electron. The coding and documentation for this software-building framework is archived at electronjs.org/docs, the contents of which are expressly incorporated herein by reference.

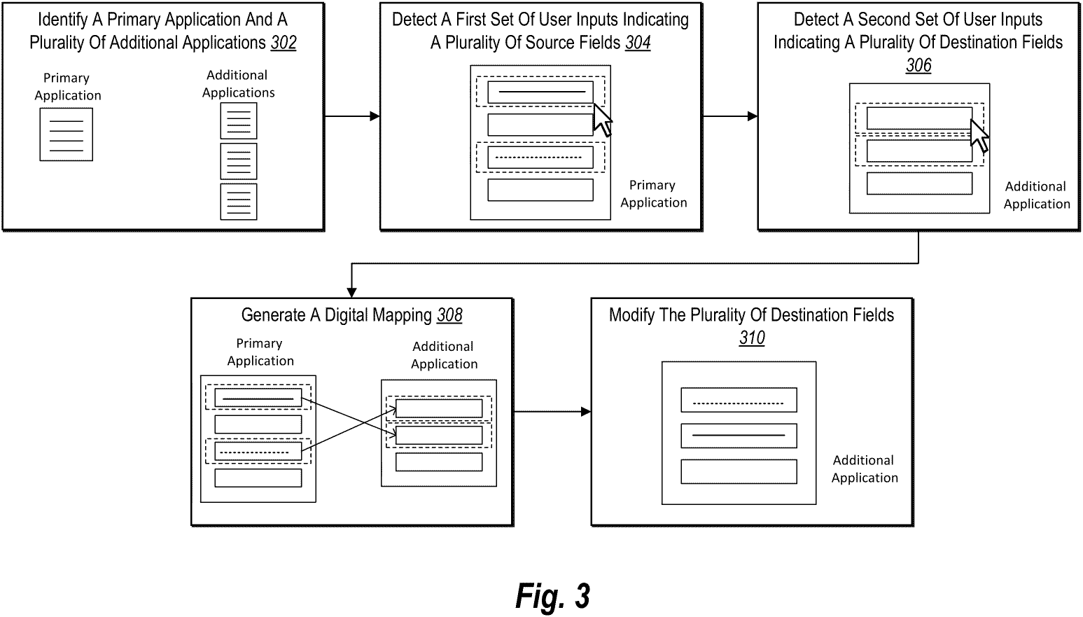

As mentioned above, the switchboard management system 104 can dynamically update additional applications based on digital mappings with primary applications. FIG. 3 illustrates the switchboard management system 104 using a digital mapping to modify a plurality of destination fields in accordance with one or more embodiments. As shown, at an act 302, the switchboard management system 104 identifies a primary application and a plurality of additional applications. By identifying these applications specifically, the switchboard management system 104 can provide a customized user interface for launching and accessing the primary application and the additional applications together. More particularly, identifying the primary application and the plurality of additional applications allows the switchboard management system 104 to determine which application comprises source fields with ground truth digital data entries and which application comprises destination fields.

There are a variety of ways to identify the primary application and the plurality of additional applications. In some embodiments, the switchboard management system 104 provides a request for user input to indicate or select the primary application and/or the plurality of additional applications. For example, the switchboard management system 104 may surface a prompt requesting that the client device launch the primary application and/or the plurality of additional applications. As another example, the switchboard management system 104 may surface a prompt requesting that the client device select the primary application and/or the plurality of additional applications from a list of applications downloaded onto the client device (or otherwise accessible via the client device). In certain implementations, the switchboard management system 104 only requests user input to identify the primary application because all other applications can correspond to the plurality of additional applications.

In other embodiments, the switchboard management system 104 suggests or predicts the primary application and/or the plurality of additional applications. For example, in certain implementations, the switchboard management system 104 uses a machine-learning model to predict the primary application and/or the plurality of additional applications. In this example, the switchboard management system 104 can use machine-learning model trained to analyze digital actions (e.g., frequency of digital actions, sequences of digital actions, etc.) and generate classifications based on probability values for various applications. Specifically, based on an application satisfying one or more threshold classification scores, the switchboard management system 104 can predict the primary application and/or the plurality of additional applications.

Still, in other embodiments, the switchboard management system 104 comprises default settings indicating a default primary application and/or default additional applications. In certain embodiments, the default settings are specific to an organization, facility, or department. In these or other cases, the default settings may be changed as desired.

At an act 304, the switchboard management system 104 detects a first set of user inputs indicating a plurality of source fields. As mentioned previously, the plurality of source fields correspond to ground truth digital data entries within the primary application that the switchboard management system 104 can use to efficiently modify destination fields (discussed below). Accordingly, identifying the plurality of source fields allows the switchboard management system 104 to identify which fields in the primary application are applicable or relevant for subsequent data transfers.

In particular embodiments, the switchboard management system 104 can detect a variety of different user inputs to identify the plurality of source fields. For example, the switchboard management system 104 can detect indications of cursor clicks, highlights, tool selections, etc. As additional examples, the switchboard management system 104 can detect haptic inputs, such as taps, swipes, long-presses, or other types of touch inputs. Further examples of user inputs include voice commands, alphanumeric inputs (e.g., “field 1,” “birthday,” etc.), or keystroke selections (e.g., “tab” and “return”).

In one or more embodiments, the switchboard management system 104 detects the first set of user inputs in response to a first set of prompts. For example, as will be discussed below in relation to FIGS. 4B-4D, the switchboard management system 104 can interactively guide user selections of specific source fields. In this manner, the switchboard management system 104 can determine the plurality of source fields in an organized manner (e.g., an arranged or sequential order).

At an act 306, the switchboard management system 104 detects a second set of user inputs indicating a plurality of destination fields. In particular embodiments, the plurality of destination fields include a set of relevant fields within a single additional application of the plurality of additional applications. Specifically, the plurality of destination fields include the particular fields of the additional application where the switchboard management system 104 will provide ground truth digital data entries from the primary application.

As with the plurality of source fields, the switchboard management system 104 can also detect a variety of user inputs indicating the plurality of destination fields. Indeed, the switchboard management system 104 can detect the same or similar user inputs described above. Moreover, in certain implementations, the switchboard management system 104 interactively prompts the client device to provide the second set of user inputs for selecting the plurality of destination fields (e.g., in a same order as done for the plurality of source fields). Such an interactive approach for selecting the plurality of destination fields is discussed further below in relation to FIGS. 4E-4F.

At an act 308, the switchboard management system 104 generates a digital mapping. In some embodiments, the digital mapping assigns the plurality of source fields of the primary application to the plurality of destination fields of a specific additional application. In these instances, the digital mapping therefore represents a one-to-one relationship of source fields to destination fields for the primary application and the additional application. Moreover, by generating the digital mapping, the switchboard management system 104 can automatically transfer data from the primary application to the specific additional application (e.g., going forward for different data records). That is, the switchboard management system 104 can use the same digital mapping to transfer different ground truth digital data entries to the specific additional application, depending on the data record accessed via the primary application.

To generate the digital mapping, the switchboard management system 104 associates the plurality of source fields with the plurality of destination fields. In particular embodiments, the switchboard management system 104 generates mapping pairs based on the first set of user inputs and the second set of user inputs. For example, in certain implementations, the switchboard management system 104 identifies or assigns a first set of field identifiers corresponding to the plurality of source fields based on the first set of user inputs. Similarly, the switchboard management system 104 can identify or assign a second set of field identifiers corresponding to the plurality of destination fields based on the second set of user inputs. In turn, one or more embodiments of the switchboard management system 104 generate mapping pairs of field identifiers for the first set of field identifiers and the second set of field identifiers. Additional detail regarding digital mapping is provided below in relation to at least FIG. 4F.

At an act 310, the switchboard management system 104 modifies the plurality of destination fields. In particular embodiments, the switchboard management system 104 modifies the plurality of destination fields to include ground truth digital data entries from the plurality of source fields. In so doing, the switchboard management system 104 can efficiently and accurately sync information from the primary application to the additional application.

To modify the plurality of destination fields, a variety of approaches may apply. In one example, the switchboard management system 104 populates a digital clipboard with the ground truth digital data entries from the plurality of source fields. In turn, the switchboard management system 104 transfers the ground truth digital data entries from the digital clipboard into the plurality of destination fields.

In another example, the switchboard management system 104 foregoes a digital clipboard. Instead, the switchboard management system 104 uses one or more processing features to duplicate, within the plurality of destination fields, the ground truth digital data entries from the primary application. For example, the switchboard management system 104 may copy the ground truth digital data entries in the plurality of source fields and then transfer directly into the plurality of destination fields by executing certain digital commands (e.g., computer code) that circumvent a digital clipboard.

In some embodiments, the switchboard management system 104 implements additional or alternative acts than just discussed. For example, in certain implementations, the switchboard management system 104 repeats at least the acts 304-308 to generate an additional digital mapping for another (e.g., a second) additional application. As previously mentioned, each digital mapping comprises a one-to-one relationship between the primary application and a given additional application. Thus, to generate a digital mapping for a second additional application, the switchboard management system 104 can detect another set of user inputs via the primary application that indicate a plurality of source fields (which may be the same or different from the plurality of source fields discussed above). Similarly, the switchboard management system 104 can detect another set of user inputs via the second additional application that indicate a plurality of destination fields.

Based on these additional user inputs, one or more embodiments of the switchboard management system 104 generate an additional digital mapping between the primary application and the second additional application (as discussed above). Moreover, in certain implementations, the switchboard management system 104 further modifies the plurality of destination fields in the second additional application to include ground truth digital data entries from plurality of source fields in the primary application based on the additional digital mapping.

In another example alternative embodiment, the switchboard management system 104 does not utilize an interactive approach to determine the plurality of source fields and/or the plurality of destination fields. For instance, in certain implementations, the switchboard management system 104 utilizes a machine-learning model to generate a digital mapping independent of user inputs. In these instances, the machine-learning model may include a digital mapping prediction model trained to generate a predicted digital mapping based on available fields (e.g., the fields comprising the primary application and the fields comprising the additional application). In other instances, the switchboard management system 104 can access, download, or otherwise retrieve a pre-generated digital mapping specific to the primary application and the additional application (e.g., from a data store, user interface menu options, etc.).

As mentioned above, the switchboard management system 104 can dynamically integrate a primary application (e.g., a system of record) and a variety of additional applications (e.g., supporting applications) within a unified platform. Through this unified platform (e.g., a switchboard application), the switchboard management system 104 can interactively guide users via user interface prompts to indicate which fields are source fields in the primary application and which fields are destination fields in an additional application. Based on these user inputs, the switchboard management system 104 can afterwards automatically copy information from different data records in the primary application to an opened additional application that is mapped to the primary application.

FIGS. 4A-4K illustrate the switchboard management system 104 providing user interfaces 402a-402k on a computing device 400 in accordance with one or more embodiments. In particular, FIG. 4A illustrates the switchboard management system 104 providing the user interface 402a of an example switchboard application for display on the computing device 400. In some embodiments, the user interface 402a is a default window or home page of the switchboard application.

As shown, the user interface 402a comprises a unified window for launching and accessing a primary application 404 and additional applications 406-410 via user interactions. Specifically, the primary application 404 corresponds to “Website A” or “healthcareexample.com,” the additional application 406 corresponds to “Application 1,” the additional application 408 corresponds to “Website B” or “EZtestform.com,” and the additional application 410 corresponds to “Application 2.” However, it can be appreciated that the primary application 404 and the additional applications 406-410 can include myriad different combinations of applications (and types of applications). In addition, the user interface 402a can include more or fewer applications than illustrated.

Further shown in FIG. 4A, the user interface 402a includes a listing 412 that details a plurality of source fields for the primary application 404. In certain implementations, the switchboard management system 104 generates the listing 412 in response to detecting user inputs for identifying the plurality of source fields (e.g., as will be described below in relation to FIGS. 4B-4D). Additionally, the user interface 402a shows the additional applications 406-410 currently have no fields configured. However, the switchboard management system 104 can generate listings similar to the listing 412 in response to detecting user inputs for identifying corresponding destination fields (e.g., as will be described below in relation to FIGS. 4E-4F).

Also shown in FIG. 4A, the user interface 402a comprises a web interface. For example, a tab 414a for the switchboard application is currently opened (albeit additional or alternative tabs for opened applications may be displayed together). Further, the user interface 402a comprises web browser functions for navigating the Internet or an intranet. Specifically, the user interface 402a comprises navigation buttons 416 (e.g., forward and backward navigation buttons) and an address bar 418 (e.g., for entering in and navigating to a universal resource locator).

In FIGS. 4B-4D, the switchboard management system 104 provides the user interfaces 402b-402d for configuring the primary application 404 by identifying the plurality of source fields. In FIG. 4B in particular, the switchboard management system 104 provides the user interface 402b in response to a user input with respect to the primary application 404 in FIG. 4A. Specifically, the switchboard management system 104 opens a new tab 414b and launches the primary application 404 comprising a login page with an authorization box 420. Via the authorization box 420, users can enter their personal username (ID) and password to access an account of the primary application 404.

As shown in FIG. 4B, the switchboard management system 104 provides a prompt 422a. The prompt 422a requests that users navigate to a page within the primary application 404 that includes fields for indicating as the plurality of source fields. Upon arriving at the desired page within the primary application 404, the prompt 422a requests a user selection of the “Start” button in the prompt 422a to initiate a digital mapping process corresponding to the primary application.

In FIG. 4C, the user interface 402c includes a particular page within the primary application 404 that includes fields for indicating as a plurality of source fields. Specifically, the user interface 402c includes a data record 424a (e.g., an electronic health record for a patient “Patient X”).

Further shown, the switchboard management system 104 provides a prompt 422b. Specifically, in response to a user selection of the “Start” button shown in the prompt 422a, the prompt 422b requests that the user provide a user input to select a field corresponding to “Patient ID.” In addition, the prompt 422b provides navigation options “Back,” “Skip,” and “Start Over” for navigating the digital mapping process.

In some embodiments, the switchboard management system 104 utilizes a variety of approaches to generate prompts like the prompt 422b with requests for selecting a specific field (e.g., “Patient ID”). In some embodiments, the switchboard management system 104 determines the specific fields to request in the prompts based on user-generated data. For example, in certain implementations, the listing 412 shown in FIG. 4A is a user-generated listing of the desired fields to map as source fields. In this example, the switchboard management system 104 uses the user-generated data in the listing 412 to generate the prompts, such as the prompt 422b.

In certain embodiments, the specific fields to request in the prompts are custom-programmed into the switchboard management system 104. For example, in one or more embodiments, the switchboard management system 104 includes the specific fields to request in the prompts based on pre-determined data that is specific to a customer, client, organization, department, or facility.

In other embodiments, the switchboard management system 104 determines the specific fields to request in prompts based on automatically identifying data (e.g., within the application page at which the digital mapping is initiated). For example, in some embodiments, the switchboard management system 104 performs a data recognition act to assign or identify each field included in an application page. To illustrate, in certain implementations, the switchboard management system 104 performs one or more data mining algorithms to scrape data from the application page (e.g., in the data record 424a), thereby identifying source field identifiers such as “First Name,” “Last Name,” “DOB,” “Birth Sex,” “Patient ID,” “SSN,” “Alerts,” and “Allergies.” In this automatic approach, the switchboard management system 104 may determine a specific field to request in a prompt that is inapplicable, unnecessary, or incorrect. In these cases, the switchboard management system 104 provides the option to “Skip” the field and thereby exclude the field from the digital mapping.

In certain implementations, the switchboard management system 104 uses other automatic approaches for identifying specific fields to request in prompts. For example, in one or more embodiments, the switchboard management system 104 uses a machine-learning model trained to predict specific fields to request in prompts based on historical user interactions with fields (e.g., clicks, copy/paste data, etc.).

With respect to FIG. 4C in particular, the user interface 402c shows the switchboard management system 104 highlighting a source field 426a in response to a user interaction (e.g., a cursor click or highlight). As depicted, the source field 426a corresponds to “Patient ID” as requested in the prompt 422b. Moreover, in accordance with the user interaction, the switchboard management system 104 can associate a specific field ID of “Patient ID” to the source field 426a.

Similarly, in FIG. 4D, the switchboard management system 104 provides the user interface 402d comprising a prompt 422c. Like the prompt 422b, the prompt 422c requests that the user provide a user interaction to indicate a specific field. Specifically, the prompt 422c requests selection of a field corresponding to “Date of Birth.”

Responsive to a user interaction, the user interface 402d shows the switchboard management system 104 highlighting a source field 426b. In particular embodiments, the switchboard management system 104 utilizes the user input (e.g., a cursor click or highlight) for correspondingly associating the source field 426b for “DOB” and the requested field in the prompt 422c for “Date of Birth.”

As part of the digital mapping process, one or more embodiments of the switchboard management system 104 map source fields to destination fields as mentioned previously. In particular embodiments, the switchboard management system 104 generates the digital mapping by identifying or assigning a first set of field identifiers corresponding to the plurality of source fields (e.g., the source fields 426a, 426b) based on the first set of user inputs.

In certain embodiments, the switchboard management system 104 assigns the source field an identifier corresponding to the identifier used in the user interface prompts. For example, in FIG. 4C, the switchboard management system 104 may assign the source field 426a the identifier “Patient ID” as requested in the prompt 422b. Similarly, in FIG. 4D, the switchboard management system 104 may assign the source field 426b the identifier “Date of Birth” as requested in the prompt 422c.

In other embodiments, the switchboard management system 104 assigns the source field an identifier corresponding to alphanumeric data corresponding to the source field itself. For example, in FIG. 4C, the switchboard management system 104 may assign the source field 426a the identifier “Patient ID” as a label already corresponding to the source field 426a. Similarly, in FIG. 4D, the switchboard management system 104 may assign the source field 426b the identifier “DOB” as illustrated for the source field 426b.

Still in other embodiments, the switchboard management system 104 identifies or assigns the source field an identifier according to non-visual elements that are specific to the source field. For example, in one or more embodiments, the switchboard management system 104 provides identifiers that specifically correspond to the source fields 426a, 426b based on html tags, source code, database labels, etc.

To complete the digital mapping process, the switchboard management system 104 configures an additional application. FIGS. 4E-4F illustrate the switchboard management system 104 providing user interfaces 402e-402f for configuring the additional application 408 (EZtestform.com).

As shown in FIG. 4E, the switchboard management system 104 provides the user interface 402e in response to a user input with respect to the additional application 408 in FIG. 4A. Specifically, the switchboard management system 104 opens a new tab 414c and launches the additional application 408 comprising a patient form with a plurality of destination fields 428.

Moreover, the switchboard management system 104 provides the user interface 402e with a prompt 422d. Similar to the prompt 422a, the prompt 422d requests that users navigate to a page within the additional application 408 that includes fields for indicating as the plurality of destination fields. Upon arriving at the desired page within the additional application 408, the prompt 422d requests a user selection of the “Start” button in the prompt 422d to initiate a digital mapping process on the additional application side.

In FIG. 4F, the switchboard management system 104 provides the user interface 402f for identifying the plurality of destination fields. In particular, the switchboard management system 104 provides a prompt 422e. Specifically, in response to a user selection of the “Start” button shown in the prompt 422d, the prompt 422e requests that the user provide a user input to select a field corresponding to “Patient ID.”

Additionally, the switchboard management system 104 provides the user interface 402f highlighting a destination field 428a in response to detecting the requested user interaction, such as a cursor click or highlight. Based on the user interaction, one or more embodiments of the switchboard management system 104 then associate the destination field 428a with an identifier such as “Patient ID,” “ID,” or another suitable identifier.

Moreover, FIG. 4F illustrates a digital mapping 430. The digital mapping 430 (denoted in dashed lines) is not necessarily visible within a user interface but is shown within the user interface 402f for explanation purposes. In particular, the switchboard management system 104 generates the digital mapping 430 by generating mapping pairs 432. To illustrate, the switchboard management system 104 generates the mapping pairs 432 by populating a data structure (e.g., an index, vector, table, nodal graph, data tree, etc.) with digital representations for source fields that correspond to digital representations for destination fields.

For example, as depicted in FIG. 4F, each mapping pair of the mapping pairs 432 comprises a field identifier (or field ID) of a source field in the primary application 404 that maps to a field ID of a destination field in the additional application 408. For instance, the first mapping pair of the mapping pairs 432 comprises the “Patient ID” field ID in the primary application 404 being paired with the “ID” field ID in the additional application 408. By pairing field identifiers in the mapping pairs 432, the switchboard management system 104 can generate the digital mapping 430 such that source fields in the primary application 404 and destination fields in the additional application 408 are linked together (e.g., for efficient, accurate data transfer).

In one or more embodiments, the switchboard management system 104 generates the mapping pairs 432 in an interactive manner. For example, as the switchboard management system 104 detects user inputs to identify source fields and destination fields, the switchboard management system 104 generates (or populates) more of the mapping pairs 432. For instance, although not illustrated in FIG. 4F, the switchboard management system 104 can subsequently detect user inputs to identify more destination fields and therefore determine corresponding field IDs for adding to the mapping pairs 432.

In some embodiments, the digital mapping 430 comprises various features and functionality. To illustrate, in certain implementations, the digital mapping 430 is editable (and visible). For example, via one or more user interface elements and/or user interactions, the switchboard management system 104 can add, remove, or revise the mapping pairs 432. In some implementations, the digital mapping 430 is also directional. For instance, in one or more embodiments, the digital mapping 430 is unidirectional (e.g., data flows only one-way, such as from source fields to destination fields). In other instances, the switchboard management system 104 generates the digital mapping 430 in a bidirectional manner such that data can flow both ways between source fields and destination fields.

After the switchboard management system 104 generates the digital mapping for the primary application 404 and the additional application 408, no further remapping between these applications is needed. In particular embodiments, the switchboard management system 104 utilizes a digital clipboard for data transfers. For example, as illustrated in FIG. 4G, the switchboard management system 104 provides a user interface 402g with a digital clipboard 434 automatically populated with ground truth digital data entries 436a-436e.

Specifically, upon accessing the data record 424a via the primary application 404, the switchboard management system 104 extracts the ground truth digital data entries 436a-436e from the plurality of source fields (e.g., the source fields 426a, 426b, and others) identified in relation to FIGS. 4B-4D. In certain implementations, the switchboard management system 104 accesses the source fields associated with the field IDs in the digital mapping 430 and copies the corresponding ground truth digital data entries into the digital clipboard 434. For instance, the switchboard management system 104 accesses each source field according to the digital mapping 430, selects the ground truth digital data entry inside the source field, and performs a copy operation of the selected contents. In turn, the switchboard management system 104 transfers the ground truth digital data entry into the digital clipboard 434 (e.g., with a corresponding field identifier).

Additionally shown, the switchboard management system 104 can clear the digital clipboard 434. For example, in response to a user interaction with the clear button 438, the switchboard management system 104 can clear the ground truth digital data entries 436a-436e from the digital clipboard 434. Additionally or alternatively, the switchboard management system 104 can clear the digital clipboard 434 in other instances which are described further below.

Once copied into the digital clipboard 434, the switchboard management system 104 can transfer the ground truth digital data entries 436a-436e to the additional application 408. In one example, the switchboard management system 104 automatically populates the destination fields 428 in the additional application 408 without navigational inputs to switch from the primary application 404 to the additional application 408. That is, the switchboard management system 104 can maintain the user interface 402g of the primary application 404 as the active window, while simultaneously modifying the destination fields 428 of the additional application 408 to include the ground truth digital data entries 436a-436e.

In another example shown in FIG. 4H, the switchboard management system 104 transfers the ground truth digital data entries 436a-436e from the digital clipboard 434 to the additional application 408 in response to accessing the additional application 408. For instance, in response to a user selection of the tab 414c, the switchboard management system 104 navigates to the additional application 408 and provides the user interface 402h. In particular, the switchboard management system 104 provides the user interface 402h with the ground truth digital data entries 436a-436e automatically populated within the destination fields 428a-428e.

In particular embodiments, the switchboard management system 104 automatically populates the destination fields 428a-428e with the ground truth digital data entries 436a-436e according to the digital mapping 430. For example, the switchboard management system 104 transfers the ground truth digital data entries 436a-436e into the destination fields 428a-428e based on corresponding field IDs for the destination fields 428a-428e that map to the field IDs of source fields in the digital mapping 430 (shown in FIG. 4F). To illustrate, the switchboard management system 104 transfers the ground truth digital data entry 436a to the destination field 428a because the ground truth digital data entry 436a corresponds to a source field 426a. The source field 426a has a field ID “Patient ID” that maps to the field ID “ID” of the destination field 428a. Therefore, the switchboard management system 104 transfers the ground truth digital data entry 436a to the destination field 428a.

In one or more embodiments, the switchboard management system 104 can also edit the digital clipboard 434. For example, the switchboard management system 104 can edit the digital clipboard 434 in response to user interaction with the digital clipboard 434 directly (if shown) or else one of the destination fields 428 (e.g., a right-click to activate or make the digital clipboard 434 visible for editing). Based on the edit(s) to the digital clipboard 434, the switchboard management system 104 can automatically update the destination fields 428 as applicable (and in some instances, the source fields as well). Moreover, by being able to edit the digital clipboard 434 within the user interface 402h of the additional application 408, the switchboard management system 104 can avoid navigating back to the primary application 404 to edit a corresponding source field in order to change a specific destination field.

As mentioned previously, the switchboard management system 104 can clear the digital clipboard 434 in response to detecting various user interactions. In one or more embodiments, the switchboard management system 104 deletes the ground truth digital data entries 436a-436e from the digital clipboard 434 in response to detecting a user interaction to navigate away from a data record in the primary application 404. In this manner, the switchboard management system 104 can maintain data security and prevent incidental/incorrect data transfers.

In certain implementations, navigating away from a data record in the primary application 404 can include exiting or logging out of the primary application 404, closing a data record, or navigating to a different page within the primary application 404. Additionally or alternatively, navigating away from a data record in the primary application 404 can include accessing a different data record. Accordingly, in one or more embodiments, the switchboard management system 104 clears the digital clipboard 434 when accessing a new or different data record (e.g., a data record 424b as discussed later in relation to FIG. 4J). It can be appreciated that numerous other triggers for clearing the digital clipboard 434 may apply.

FIG. 4I illustrates one such example. In particular, the switchboard management system 104 provides the user interface 402i of the primary application 404 in response to a user interaction to navigate away from the data record 424a and access a patient search portal 440 (e.g., to search for patient “749322”). In response to navigating to the patient search portal 440, the user interface 402i shows the switchboard management system 104 clearing the digital clipboard 434 by deleting the ground truth digital data entries 436a-436e from the digital clipboard 434.

Additionally or alternatively to clearing the digital clipboard 434, in one or more embodiments, the switchboard management system 104 clears the ground truth digital data entries 436a-436e from the additional application 408. Indeed, responsive to one or more of the above triggers for clearing the digital clipboard 434, the switchboard management system 104 can also delete the ground truth digital data entries 436a-436e from the additional application 408 such that the destination fields 428a-428e in FIG. 4H are cleared. Although not illustrated, this approach can also improve security and/or privacy measures (e.g., by helping to avoid incidental/incorrect data transfers).

As also mentioned above, the switchboard management system 104 can use the digital mapping 430 to automatically transfer data from the primary application 404 to the additional application 408 for a variety of different data records. Indeed, FIG. 4J illustrates the switchboard management system 104 accessing a data record 424b and correspondingly populating the digital clipboard 434 (previously cleared). Specifically, the user interface 402j shows the switchboard management system 104 automatically populating the digital clipboard 434 with ground truth digital data entries from the data record 424b.

To populate the digital clipboard 434 with ground truth digital data entries from the data record 424b, the switchboard management system 104 uses the digital mapping 430. In particular, the switchboard management system 104 identifies the source fields by corresponding field IDs in the digital mapping 430. In turn, the switchboard management system 104 copies the ground truth digital data entries in the source fields and pastes the ground truth digital data entries into the digital clipboard 434.

Upon populating the digital clipboard 434, FIG. 4K illustrates the switchboard management system 104 accessing the additional application 408 and providing a user interface 402k comprising the additional application 408 automatically synced with the digital clipboard 434. For instance, in response to a user selection of the tab 414c, the switchboard management system 104 navigates to the additional application 408 and pastes in the ground truth digital data entries 436a-436e from the digital clipboard 434 into the destination fields 428a-428e. Specifically, the switchboard management system 104 transfers the ground truth digital data entries 436a-436e into the destination fields 428a-428e based on corresponding field IDs for the destination fields 428a-428e that map to the field IDs of source fields in the digital mapping 430. In this manner, the switchboard management system 104 provides a powerful, efficient data transfer approach that flexibly applies to many different data records.

Additionally or alternatively to one or more of the user interfaces 402a-402k, myriad different presentation forms of display may apply. For example, in certain implementations, the switchboard management system 104 opens one of the primary application 404 or the additional applications 406-410 for display by default (e.g., as opposed to showing the user interface 402a by default).

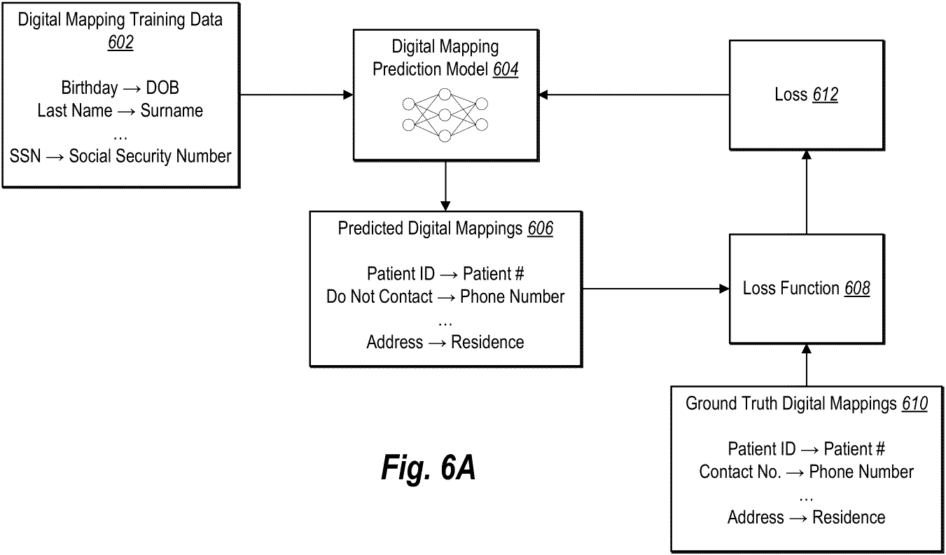

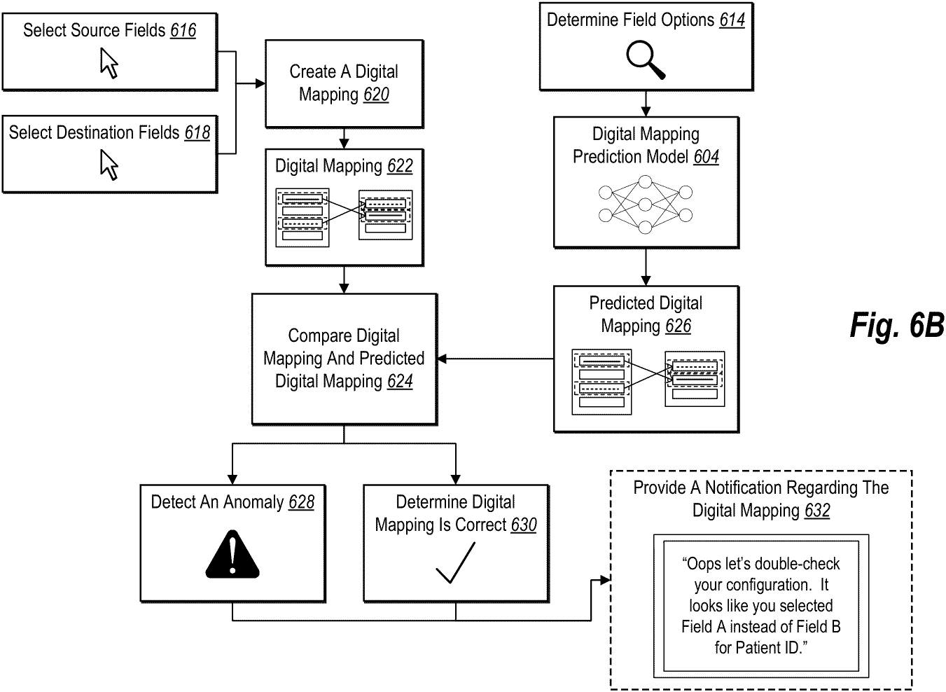

As another example, the switchboard management system 104 may optionally provide prompts for selecting a field (or multiple fields at a time), but not a specific field. In this example, the switchboard management system 104 uses optical character recognition, machine-learning, etc. to determine or predict which particular field(s) were selected. Similarly, the switchboard management system 104 can automatically generate a predicted digital mapping (without any user inputs) as described below in relation to FIGS. 6A-6B. Under these approaches, the switchboard management system 104 can reduce incorrect digital mappings based on incorrect user inputs (e.g., a user input indicating a first name field corresponds to a requested date of birth field).

As mentioned above, the switchboard management system 104 can provide a variety of additional or alternative features. For example, in certain implementations, the switchboard management system 104 can audit selected activity codes by comparing predicted activity codes from a machine-learning model. In turn, the switchboard management system 104 can provide a notification (e.g., a warning message, error alert, etc.) regarding a selected combination of activity codes.

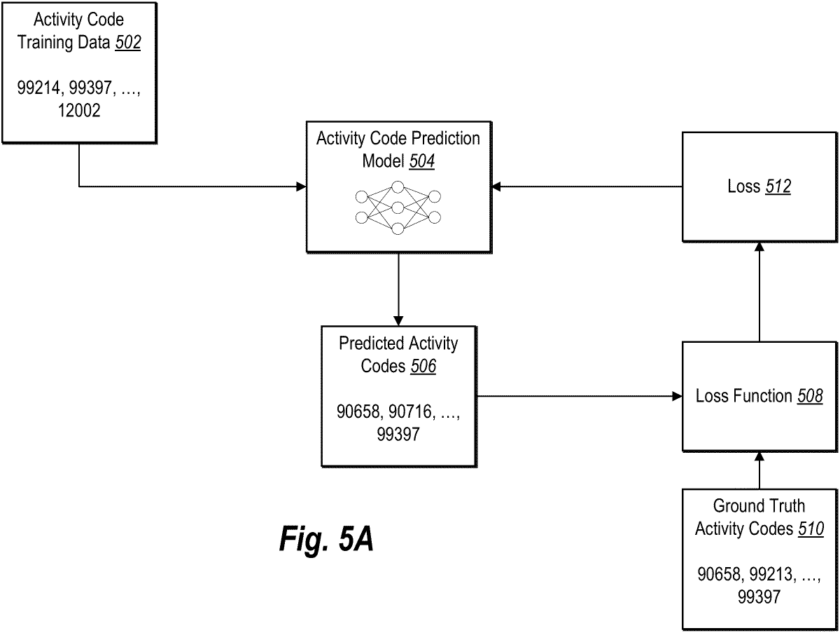

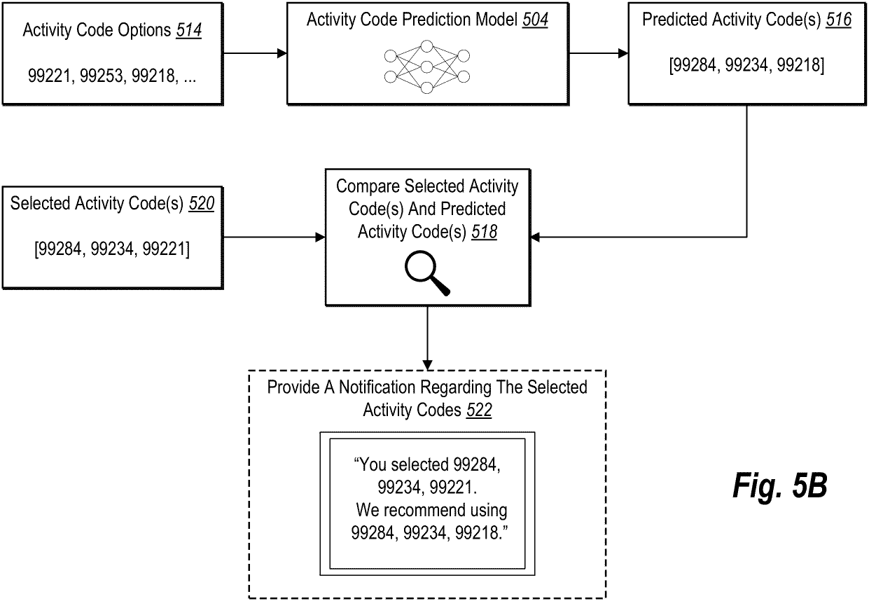

FIGS. 5A-5B illustrate the switchboard management system 104 training and implementing an activity code prediction model 504 in accordance with one or more embodiments. As shown in FIG. 5A, the switchboard management system 104 trains the activity code prediction model 504 based on activity code training data 502. In some embodiments, the activity code training data 502 can include activity code options, such as lists or sequences of activity codes (e.g., numbers, letters, punctuation, and/or other characters combined together) that digitally represent particular activities. For example, in one or more embodiments, the activity code training data 502 includes a plurality of different sets of activity code options (e.g., current procedural terminology (CPT) codes that represent one or more discrete activities or elements of a medical procedure).

In one or more embodiments, the switchboard management system 104 generates the activity code training data 502 by generating predetermined sets of activity code options. For example, in some embodiments, the switchboard management system 104 randomly generates the predetermined sets of activity code options. Additionally or alternatively, the switchboard management system 104 accesses a database or computing network to retrieve actual sets of activity code options, such as different sets possible of CPT codes for a plurality of different medical procedures performed over a given time period and in various organization departments. Still further, in some embodiments, the switchboard management system 104 improves a robustness of the activity code prediction model 504 by generating the activity code training data 502 with applied errors (e.g., based on ground truth activity codes with extra characters, different characters, mixed characters, and/or removed characters).

Based on the activity code training data 502, the switchboard management system 104 utilizes the activity code prediction model 504 to generate predicted activity codes 506. To do so, the activity code prediction model 504 processes a variety of different inputs that comprise the activity code training data 502. For example, in some embodiments, the switchboard management system 104 processes the activity code training data 502 comprising a variety of different features that correspond to a procedure. To illustrate, the activity code training data 502 can include classifications, labels, notes, voice memos, scanned barcodes of medications, instructions, records corresponding to the procedure, activity codes selected by an administrator device for the procedure, all possible activity codes corresponding to the procedure, etc. In certain implementations, the activity code training data 502 comprises encoded data corresponding to the above example features for the procedure (e.g., word embeddings, sentence embeddings, tokens, vector-based representations, hash values, etc.). In turn, the activity code prediction model 504 can process this encoded data (e.g., as generated by an embedding model) in order to generate the predicted activity codes 506 for the procedure.

A variety of machine-learning models can perform this act. For example, the activity code prediction model 504 can include one or more of a decision tree (e.g., a gradient boosted decision tree), a linear regression model, a logistic regression model, association rule learning, inductive logic programming, support vector learning, a Bayesian network, a regression-based model, principal component analysis, a neural network, or a combination thereof.

In one or more embodiments, the predicted activity codes 506 comprises various combinations or subsets of activity codes. For example, given a list of ten possible activity codes for a specific medical procedure in the activity code training data 502, the activity code prediction model 504 generates the predicted activity codes 506 comprising a subset of the ten possible activity codes (e.g., three, five, or eight activity codes). In certain implementations, the activity code prediction model 504 generates the predicted activity codes 506 comprising combinations of activity codes that are predicted as corresponding to actual (e.g., performed or completed) aspects of a procedure.

In turn, the switchboard management system 104 compares the predicted activity codes 506 and ground truth activity codes 510 utilizing a loss function 508. In particular embodiments, the ground truth activity codes 510 correspond to actual aspects of a procedure or activity codes previously determined as accurate, optimal, or preferred. Based on this comparison, the switchboard management system 104 generates a loss 512.

Examples of a loss function 508 can include a regression loss function (e.g., a mean square error function, a quadratic loss function, an L2 loss function, a mean absolute error/L1 loss function, mean bias error, etc.). Additionally or alternatively, the loss function 508 can include a classification loss function (e.g., a hinge loss/multi-class SVM loss function, cross entropy loss/negative log likelihood function, etc.). Further, the loss function 508 can return the loss 512 comprising quantifiable data regarding the difference between the predicted activity codes 506 and the ground truth activity codes 510. In particular, the loss function 508 can return the loss 512 to the activity code prediction model 504 where the switchboard management system 104 can adjust various parameters/weights to improve the quality/accuracy of the predicted activity codes 506 by reducing the loss (e.g., via backpropagation). Moreover, the training/learning of the activity code prediction model 504 can be an iterative process (as shown by the return arrow between the loss function 508 and the activity code prediction model 504) such that the switchboard management system 104 can continually adjust parameters/hyperparameters of the activity code prediction model 504 over learning cycles.

In alternative embodiments, the switchboard management system 104 can use other algorithms, such as optimization algorithms that maximize and/or minimize one or more metrics (e.g., revenue, costs), a reward, customer satisfaction, etc. In this example, the switchboard management system 104 uses an optimization algorithm that processes a predetermined set of activity codes (e.g., activity code options for a given procedure) based on optimized weights or tuned parameters to determine an optimal set of activity code options.

In FIG. 5B, the switchboard management system 104 utilizes the activity code prediction model 504 to automatically generate predicted activity code(s) 516. For example, as similarly described above, the activity code prediction model 504 processes activity code options 514 to generate the predicted activity code(s) 516. To illustrate, activity code prediction model 504 processes activity code options “99221,” “99253,” “99218” and others. Based on processing these activity code options, the activity code prediction model 504 generates the predicted activity code(s) 516 comprising a set of three activity codes “99284,” “99234,” and “99218.”

Additionally shown, at an act 518 the switchboard management system 104 compares selected activity code(s) 520 and the predicted activity code(s) 516. In one or more embodiments, the act 518 comprises determining matching activity codes between the selected activity code(s) 520 and the predicted activity code(s) 516. For instance, the switchboard management system 104 can analyze individual characters and/or sequences of characters to determine whether activity codes are the same or different from each other. To illustrate, in certain implementations, the switchboard management system 104 analyzes each character of one of selected activity code(s) 520 and then searches for a matching sequence of characters in the predicted activity code(s) 516. If the switchboard management system 104 determines that none of the predicted activity code(s) 516 comprise a matching sequence of characters, then the particular activity code of the selected activity code(s) 520 may include a potential error or unfavorable selection.

For example, the selected activity code(s) 520 includes activity codes “99284,” “99234,” and “99221.” Therefore, the switchboard management system 104 can determine that the selected activity code “99221” does not match the predicted activity code “99218” (e.g., based on corresponding sequences of characters). Alternatively, in some cases, the switchboard management system 104 can determine that the selected activity code(s) 520 does match the predicted activity code(s) 516.

At an act 522, the switchboard management system 104 provides a notification regarding the selected activity code(s) 520. For example, in certain implementations, the switchboard management system 104 provides a warning, error alert, or recommendation in response to determining that the selected activity code(s) 520 fails to match the predicted activity code(s) 516. As another example, the switchboard management system 104 provides a confirmation notice to indicate that the selected activity code(s) 520 matches the predicted activity code(s) 516. In these or other embodiments, at the act 522 the switchboard management system 104 can provide the notice to myriad different computing devices (e.g., the client device providing the selected activity code(s) 520, an administrator device, a third-party server, etc.).

It can be appreciated that the acts illustrated in FIG. 5B may be adjusted according to different embodiments. For example, in one or more embodiments, the switchboard management system 104 automatically provides the predicted activity code(s) 516 to a client device without requiring the selected activity code(s) 520 (or any user selection of activity codes). Indeed, in one or more embodiments, the switchboard management system 104 omits the comparison step of the act 518 altogether. Instead, in certain implementations, the switchboard management system 104 provides the predicted activity code(s) 516 for display within a user interface on a client device for user recommendation and/or approval.

As mentioned above, the switchboard management system 104 can audit digital mappings to help improve accuracy and efficiency of data transfers. FIGS. 6A-6B illustrate the switchboard management system 104 training and implementing a digital mapping prediction model 604 in accordance with one or more embodiments. As shown in FIG. 6A, the switchboard management system 104 trains the digital mapping prediction model 604 based on digital mapping training data 602.

In some embodiments, the digital mapping training data 602 comprises input pairs that the digital mapping prediction model 604 processes in order to predict whether the input pairs are accurate or not. To illustrate, the digital mapping training data 602 may include training mapping pairs such as “Birthday➔DOB,” “Last Name➔Surname,” and “SSN➔Social Security Number.” In these examples, “Birthday,” “Last Name,” and “SSN” may each correspond to a primary application, and “DOB,” “Surname,” and “Social Security Number” may each correspond to an additional application. In specific implementations, the training mapping pairs include each unique combination of field identifiers for the primary application and the additional application.

In certain embodiments, the digital mapping training data 602 comprises a collection of field options that the digital mapping prediction model 604 processes to predict which fields map to each other. To illustrate, the digital mapping training data 602 may include training field identifiers that correspond to one or more fields within primary applications and additional applications. For instance, the digital mapping training data 602 comprises a list of field identifiers for all fields of a primary application and all fields of an additional application.

In one or more embodiments, the switchboard management system 104 utilizes a variety of different approaches to generate the digital mapping training data 602. In some embodiments, the switchboard management system 104 uses automated approaches. For example, in certain implementations, the switchboard management system 104 uses one or more data mining approaches to identify field options of primary applications and/or additional applications. In additional or alternative embodiments, the switchboard management system 104 utilizes user-generated data, such as digital labels, tags, etc. For example, the user-generated data may indicate two field options are a mapping pair. Still further, in some embodiments, the switchboard management system 104 improves a robustness of the digital mapping prediction model 604 by generating the digital mapping training data 602 using flawed mapping pairs based on modifications to ground truth digital mappings 610.

Based on the digital mapping training data 602, the switchboard management system 104 utilizes the digital mapping prediction model 604 to generate predicted digital mappings 606. Similar to the activity code prediction model 504, the digital mapping prediction model 604 can include a wide variety of different machine-learning models to process the digital mapping training data 602 according to one or more preset and/or learned parameters (e.g., depending on the type of machine-learning model). Moreover, in some implementations, the digital mapping prediction model 604 generates the predicted digital mappings 606 by processing the digital mapping training data 602 based on one or more preset and/or learned parameters that account for certain metrics, such as semantic similarity between field identifiers.

In some embodiments, the switchboard management system 104 generates the predicted digital mappings 606 by using the digital mapping prediction model 604 to process input pairs. Accordingly, in certain embodiments, the predicted digital mappings 606 comprise a digital mapping composed of sufficiently accurate input pairs. For example, the digital mapping prediction model 604 can score each of the input pairs. Based on the scores satisfying a score threshold (e.g., a similarity metric), the digital mapping prediction model 604 can classify a given input pair as a mapped pair. In turn, the switchboard management system 104 can combine the mapped pairs to generate a predicted digital mapping.

Alternatively to input pairs, in some embodiments, the digital mapping prediction model 604 processes a set of source fields for a primary application and a set of destination fields for an additional application to generate the predicted digital mappings 606. For example, in certain implementations, the digital mapping prediction model 604 processes the various field options to generate the predicted digital mappings 606 comprising predicted mapping pairs of field identifiers mapped to each other. In particular embodiments, the predicted digital mappings 606 comprises predicted mapping pairs that each includes a field identifier of a source field in a primary application and a field identifier of a destination field in an additional application. For example, as shown in FIG. 6A, the predicted digital mappings 606 may include “Patient ID➔Patient #,” “Do Not Contact➔Phone Number,” and “Address➔Residence.”

Utilizing a loss function 608, the switchboard management system 104 then compares the predicted digital mappings 606 and ground truth digital mappings 610. In particular embodiments, the ground truth digital mappings 610 correspond to accurate, verified, or supplied digital mappings (and/or individual mapping pairs) for comparing with the predicted digital mappings 606. In some instances, the ground truth digital mappings 610 correspond to historically used or common digital mappings for an organization. Based on this comparison via the loss function 608, the switchboard management system 104 generates a loss 612. Like the loss function 508 described above in relation to FIG. 5A, the loss function 608 can include a wide variety of different loss functions to generate the loss 612. For example, the loss function 608 can generate the loss 612 that quantifies the difference between the incorrectly predicted digital mapping with “Do Not Contact” and the correct digital mapping with “Contact No.” Moreover, FIG. 6A shows the switchboard management system 104 providing the loss 612 to the digital mapping prediction model 604 for adjusting parameters/hyperparameters of the digital mapping prediction model 604 over one or more learning cycles.

As shown in FIG. 6B, the switchboard management system 104 utilizes the digital mapping prediction model 604 to generate a predicted digital mapping 626 (e.g., for auditing a digital mapping based on user inputs). In particular, at an act 614, the switchboard management system 104 determines field options within a primary application and an additional application. In certain embodiments, the switchboard management system 104 determines field options by using one or more data mining algorithms to scrape field identifiers corresponding to source fields and destination fields. Additionally or alternatively, in one or more embodiments, the switchboard management system 104 parses specific application pages that comprise interactive portions or fields for entering data.

In a specific example, the switchboard management system 104 uses neighbor-based parsing to parse in a vicinity around a field. For instance, in certain implementations, switchboard management system 104 only parses alphanumeric text adjacent to a field or placeholder text inside of a field. The vicinity around (or adjacent to) a field can include a threshold spacing measurement or character count from a border of the field, a predetermined shape or size region around a field, etc.

Based on the field options, the digital mapping prediction model 604 generates the predicted digital mapping 626 by predicting which source fields in the primary application map to destination fields in the additional application. The predicted digital mapping 626, like other digital mappings, comprises mapping pairs of field identifiers. Specifically, the predicted digital mapping 626 comprises predicted mapping pairs that map field identifiers between source fields and destination fields of a primary application and an additional application.

Further shown in FIG. 6B, at an act 616 the switchboard management system 104 selects source fields. In particular embodiments, the switchboard management system 104 selects source fields according to one or more user inputs indicating source fields within the primary application (as described above).

Similarly, at an act 618, the switchboard management system 104 selects destination fields. Specifically, the switchboard management system 104 the switchboard management system 104 selects destination fields according to one or more user inputs indicating destination fields within the additional application (as also described above).

At an act 620, the switchboard management system 104 creates a digital mapping 622 based on the selected source fields and destination fields. For example, as described above, the switchboard management system 104 generates mapping pairs comprising pairs of field identifiers. For instance, the switchboard management system 104 generates the digital mapping 622 with mapping pairs such that each respective mapping pair comprises an identifier for a source field and an identifier for a destination field.

At an act 624, the switchboard management system 104 compares the digital mapping 622 and the predicted digital mapping 626 (both digital mappings corresponding to a specific primary application and a specific additional application). In some embodiments, the comparison comprises comparing the mapping pairs of the digital mapping 622 and the predicted mapping pairs of the predicted digital mapping 626. For example, in one or more embodiments, the switchboard management system 104 performs a character analysis to identify and compare characters of field identifiers in the mapping pairs of the digital mapping 622 and the predicted mapping pairs of the predicted digital mapping 626. As another example, the switchboard management system 104 performs a word-based comparison. For instance, in certain implementations, the switchboard management system 104 uses one or more natural language processing algorithms or models to process each of the field identifiers in the mapping pairs of the digital mapping 622 and the predicted mapping pairs of the predicted digital mapping 626. Example natural language processing algorithms or models can include edit distance, cosine similarity, vectorization, bag of words, term frequency and inverse document frequency, text normalization, stemming and lemmatization, naïve Bayesian analysis, word embedding, long short-term memory models, etc.

Based on the comparison at the act 624, the switchboard management system 104 executes one of an act 628 or an act 630. At the act 628, the switchboard management system 104 detects an anomaly in the digital mapping 622. In certain embodiments, an anomaly in the digital mapping 622 comprises one or more mapping pairs that do not match the predicted mapping pairs of the predicted digital mapping 626. For example, depending on the comparison, an anomaly may comprise a mapping pair of the digital mapping 622 that fails to satisfy a threshold cosine similarity with a corresponding mapping pair of the predicted digital mapping 626.

In contrast, at the act 630, the switchboard management system 104 determines that the digital mapping 622 is accurate based on the comparison. In one or more embodiments, determining the digital mapping 622 is correct comprises determining that each of the mapping pairs of the digital mapping 622 match the predicted digital mapping 626. For instance, each mapping pair satisfies a threshold cosine similarity with a corresponding mapping pair of the predicted digital mapping 626.

At an act 632, the switchboard management system 104 provides a notification to a client device regarding the digital mapping 622. If an anomaly is present, the switchboard management system 104 can provide a notice informing the client device (and/or other client devices, such as an administrator device) of the anomaly. Additionally or alternatively, the switchboard management system 104 may recommend the predicted digital mapping 626 and/or how to modify the digital mapping 622 to match with the predicted digital mapping 626 (e.g., in an interactive manner via prompts).

If no anomaly is present, in certain implementations, the switchboard management system 104 provides confirmation to the client device that the digital mapping 622 is accurate. Still, in other embodiments, the switchboard management system 104 provides no notification to the client device.

Although not illustrated in FIG. 6B, the switchboard management system 104 can implement a variety of additional or alternative embodiments. For example, in some embodiments, the digital mapping prediction model 604 processes input pairs in order to generate the predicted digital mapping 626. To illustrate, in certain implementations, the digital mapping prediction model 604 processes input pairs comprising each combination of field options, such as the source fields and the destination fields determined at the act 614. Additionally or alternatively, the digital mapping prediction model 604 processes the source fields and the destination fields selected by the user at the acts 616, 618 discussed above. Based on the provided input pairs, the digital mapping prediction model 604 can score the input pairs and generate the predicted digital mapping 626 composed of accurate mapping pairs.

As another example, the digital mapping prediction model 604 processes previously generated digital mappings for a given primary application and additional application. In certain implementations, the previously generated digital mappings may correspond to digital mappings generated by one or more particular switchboard user accounts (e.g., general user accounts, administrator user accounts, customer accounts, third-party accounts, etc.). Moreover, in certain implementations, the digital mapping prediction model 604 may weight previously generated digital mappings for a specific combination of a primary application and additional application (e.g., based on a number of other user accounts using a previously generated digital mapping for the same application combination).

Additionally, it can be appreciated that one or more of the acts illustrated in FIG. 6B can be performed on a single computing device or multiple computing devices (e.g., in parallel or at different times). For example, in certain implementations, a first implementing computing device of the switchboard management system 104 performs the act 614 to determine field options and an act to generate the predicted digital mapping 626 using the digital mapping prediction model 604. Additionally, in certain implementations, a second implementing computing device performs the act 620 to create the digital mapping 622 based on user inputs. In turn, the switchboard management system 104 can compare the predicted digital mapping 626 from the first implementing computing device and the digital mapping 622 from the second implementing computing device to determine accuracy. In this manner, the switchboard management system 104 can leverage digital mappings previously generated by other implementing computing devices for a same combination of a primary application and additional application (e.g., within a same organization).

As mentioned above, the switchboard management system 104 can dynamically communicate with third-party servers. Through this communication, the switchboard management system 104 can efficiently and securely transmit (and receive) myriad types of digital content. FIG. 7 illustrates the client device 106 implementing a third-party interchange system 702 in accordance with one or more embodiments.

In one or more embodiments, the third-party interchange system 702 provides a digital interface or connection between the client device 106 and the third-party server(s) 114, thereby facilitating private (e.g., encrypted) communication between disparate computing devices and computer networks. For example, the third-party server(s) 114 correspond to one or more computing systems of other (e.g., third-party) organizations. In certain implementations, the third-party server(s) 114 implement the switchboard management system 104. In other implementations, however, the third-party server(s) 114 do not implement the switchboard management system 104.

In particular embodiments, the third-party interchange system 702 comprises a digital interface or connection between the client device 106 and the third-party server(s) 114 formed by the switchboard management system 104 providing a digital mapping (as described above) between an application of the client device 106 and an application of the third-party server(s) 114. Additionally or alternatively, the third-party interchange system 702 comprises one or more application programming interfaces, system links, plug-ins, or other digital connections between the client device 106 and the third-party server(s) 114. Accordingly, as shown in FIG. 7, the client device 106 and the third-party server(s) 114 exchange shared digital content 704 and access permissions 706 via the third-party interchange system 702. For instance, both the client device 106 and the third-party server(s) 114 can access the third-party interchange system 702 to transmit and/or receive data corresponding to the shared digital content 704 and the access permissions 706.

In some embodiments, the shared digital content 704 comprises a variety of different data. For example, the shared digital content 704 comprises recommended referrals, follow-up services, instructions, tasks, appointments, documentation, files, test results, images, records, progress charts, medical history, receipts, reports, prescriptions, etc. As additional examples, the shared digital content 704 comprises customer data, sales, website visits, research data, drawings, designs, and the like.

Moreover, in one or more embodiments, the third-party interchange system 702 allows the client device 106 and the third-party server(s) 114 to interact with the shared digital content 704. For example, in some embodiments, the client device 106 provides the shared digital content 704 comprising tasks, reminders, or alerts that the third-party server(s) 114 can accept, decline, comment on, like, approve, thank, or “check off” as completed. Similarly, in certain implementations, the client device 106 and the third-party server(s) 114 can interact with the shared digital content 704 in a collaborative manner (e.g., for viewing or editing a same digital file). For instance, in one or more embodiments, the client device 106 and the third-party server(s) 114 can each access a particular X-ray of the shared digital content 704 at a same time while also communicating in real-time via the third-party interchange system 702 through instant messaging.

In addition, both the client device 106 and the third-party server(s) 114 can access the third-party interchange system 702 to transmit and/or receive data corresponding to the access permissions 706. In some embodiments, the access permissions 706 comprises digital authorizations or rights for controlling access to, sharing, generating, and/or editing of the shared digital content 704. Further, in certain implementations, the access permissions 706 associates specific digital rights with particular user accounts (e.g., switchboard user accounts or third-party user accounts). For example, the client device 106 may provide a single user account at the third-party server(s) 114 (e.g., of a referred physician) digital rights to access the medical history of a particular patient, while other user accounts at the third-party server(s) 114 are excluded from doing so. As another example, the access permissions 706 may only permit certain user accounts to generate or update task lists, care management plans, etc.

In these or other embodiments, the third-party interchange system 702 provides secure, efficient communication between the client device 106 and the third-party server(s) 114 in a variety of ways. For example, the third-party interchange system 702 can provide different modes of communication via email, instant messaging or chat, text (SMS) messaging, application messaging, system alerts, shared calendars (or other shared files), fax, voice calling, video calling, etc.

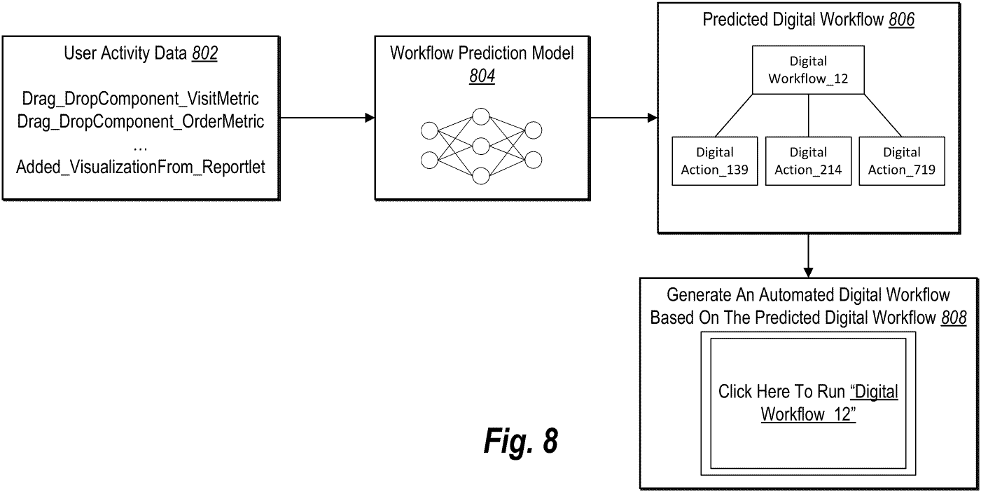

As mentioned above, the switchboard management system 104 can dynamically track user activity (e.g., digital actions) to predict digital workflows. FIG. 8 illustrates the switchboard management system 104 generating a predicted digital workflow for automating in accordance with one or more embodiments.

As shown in FIG. 8, the switchboard management system 104 provides user activity data 802 to a workflow prediction model 804. As used herein, the terms user activity or user activity data refer to an action executed using a computing device. In particular embodiments, user activity data can refer to a digital action performed by a user of a computing device, using functions and features of the computing device. For example, a user activity can include an action related to analyzing digital data (e.g., via a user interface), such as launching a project, dragging-anddropping one or more components, saving a report, clicking a node, or calculating a value. However, user activities can include an action other these examples in the context of digital workflow analysis. For example, a user activity can include an action related to clicking on a toolbar/panel option, executing a “save,” selecting a row/column of data, filtering metrics, downloading a graphical visualization, or performing a drag-and-drop operation.

To provide the user activity data 802, one or more embodiments of the switchboard management system 104 access or identify digital actions in a digital action log. In these or other embodiments, a digital action log can include a digital record that stores digital actions executed by a computing device associated with a user or user account (e.g., in response to input from the user and/or under a user profile/account associated with the user). In particular embodiments, a digital action log can include a digital record that stores a chronological list of digital actions or otherwise includes a timed record of digital actions selected by a user via one or more platforms, operating systems, computer applications, and/or user interfaces. For example, a digital action log can include raw clickstream data, such as a sequence of digital actions executed by a user via a client device (e.g., digital actions of “Drag_DropComponent_VisitMetric,” “Drag_DropComponent_OrderMetric,” and “Added_VisualizationFrom_Reportlet”).

In turn, the switchboard management system 104 utilizes the workflow prediction model 804 to process the user activity data 802. The workflow prediction model 804 may include a variety of machine-learning models (e.g., as described above for the activity code prediction model 504 and the digital mapping prediction model 604). Additional examples of the workflow prediction model 804 may include a Gaussian mixture model, a K-means clustering model, or a Spectral model. Indeed, the workflow prediction model 804 may include a machine-learning model trained to cluster, classify, or segment digital actions into predicted digital workflows. For example, the workflow prediction model 804 may process the user activity data 802 to predict or cluster digital actions that co-occur within a user session. For instance, the workflow prediction model 804 may analyze timestamps to determine repeated digital actions at certain times, intervals of times, or between timeout periods (e.g., pauses in user activity) of some threshold period of time.

Using clustered digital actions, the workflow prediction model 804 can then determine a frequency at which these clustered digital actions occur. For example, the workflow prediction model 804 can generate a histogram that maps a frequency to a cluster of digital actions. In turn, the workflow prediction model 804 can determine a cluster of digital actions comprises a digital workflow based on the cluster of digital actions satisfying a frequency threshold.

Additionally or alternatively to clustering, the workflow prediction model 804 may utilize particular algorithms or heuristics for performing frequent pattern mining to determine patterns that appear frequently within the user activity data 802 as done in certain types of analyses like market-based analyses or affinity analyses. To illustrate, the workflow prediction model 804 may implement an itemset mining algorithm, such as association rules, the Apriori algorithm, the Park-Chen-Yu (or PCY) algorithm, prefix-tree structure algorithms (also known as FP-tree based algorithms), or association rule mining.

Based on processing the user activity data 802, the workflow prediction model 804 specifically generates a predicted digital workflow 806. In some embodiments, the predicted digital workflow 806 comprises a grouping of digital actions. For example, the switchboard management system 104 generates the predicted digital workflow 806 that comprises a grouping of digital actions that frequently co-occur during a user session while logged into a switchboard account. For instance, as shown in FIG. 8, the switchboard management system 104 generates the predicted digital workflow 806 as comprising the “Digital Workflow_12” which includes three digital actions “Digital Action_139,” “Digital Action_214,” and “Digital Action_719.”

In turn, at an act 808, the switchboard management system 104 generates an automated digital workflow based on the predicted digital workflow 806. In particular embodiments, the switchboard management system 104 generates one or more macros (e.g., series of computer-executable instructions) that, when executed, perform the predicted digital workflow 806. In other embodiments, the switchboard management system 104 generates one or more user interface tools, buttons, or menu options for display within a user interface of the switchboard application to help aid execution of the predicted digital workflow 806.

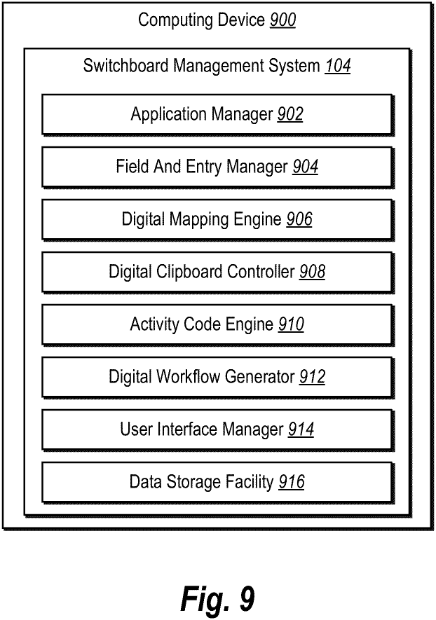

Turning to FIG. 9, additional detail will now be provided regarding various components and capabilities of the switchboard management system 104. In particular, FIG. 9 illustrates an example schematic diagram of a computing device 900 (e.g., the server(s) 102, the client device 106, the administrator device 110, the third-party server(s) 114, and/or the computing device 400) implementing the switchboard management system 104 in accordance with one or more embodiments of the present disclosure. As shown, the switchboard management system 104 includes an application manager 902, a field and entry manager 904, a digital mapping engine 906, a digital clipboard controller 908, an activity code engine 910, a digital workflow generator 912, a user interface manager 914, and a data storage facility 916.

The application manager 902 provides, analyzes, identifies, stores, transmits, and/or manages primary applications and additional applications (as described in relation to the foregoing figures). In particular embodiments, the application manager 902 identifies a primary application and a plurality of additional applications in response to user inputs indicating an application is a primary application and/or an additional application. Additionally or alternatively, the application manager 902 can use a machine-learning model to predict the primary application and/or the plurality of additional applications.

The field and entry manager 904 analyzes, identifies, compares, stores, transmits, populates, extracts contents, etc. of source fields and destination fields (as described in relation to the foregoing figures). In particular embodiments, the field and entry manager 904 identifies a plurality of source fields for a primary application based on a first set of user inputs. Similarly, the field and entry manager 904 can identify a plurality of destination fields for an additional application based on a second set of user inputs. Further, in one or more embodiments, the field and entry manager 904 can transfer ground truth digital data entries from a source application into a digital clipboard. In addition, the field and entry manager 904 can coordinate with the digital clipboard controller 908 to populate destination fields with ground truth digital data entries from the digital clipboard.

In one or more embodiments, the digital mapping engine 906 generates digital mappings (as described in relation to the foregoing figures). In particular embodiments, the digital mapping engine 906 generates mapping pairs by determining field identifiers of source fields that map to field identifiers of destination fields. In certain implementations, the digital mapping engine 906 provides digital mappings to the field and entry manager 904 and/or the digital clipboard controller 908 for determining which fields to extract, copy, or paste ground truth digital data entries. In some embodiments, the digital mapping engine 906 also audits digital mappings generated in response to user input and/or predicts digital mappings for recommendation or alert.

The digital clipboard controller 908 dynamically manages a digital clipboard (as described in relation to the foregoing figures). In particular embodiments, the digital clipboard controller 908 clears the digital clipboard in response to certain user interactions with respect to a primary application and/or additional application (e.g., to maintain data security). In addition, the digital clipboard controller 908 can populate the digital clipboard with ground truth digital data entries copied from source fields.

The activity code engine 910 can analyze, identify, or generate activity codes (as described in relation to the foregoing figures). In particular embodiments, the activity code engine 910 audits user-selected activity codes and/or automatically predicts activity codes. For example, based on determining that user-selected activity codes fail to match predicted activity codes, the activity code engine 910 can provide an alert regarding the user-selected activity codes and/or recommend the predicted activity codes.

The digital workflow generator 912 can generate predicted workflows (as described in relation to the foregoing figures). In particular embodiments, the digital workflow generator 912 utilizes a workflow prediction model to process user activity data. For example, the digital workflow generator 912 can cluster, classify, or segment digital user activities into predicted digital workflows. Based on the predicted digital workflows, the digital workflow generator 912 can generated automated workflows (e.g., macros) for automatically or semi-automatically performing within a switchboard application.

In one or more embodiments, the user interface manager 914 provides, manages, and/or controls a graphical user interface (or simply “user interface”). In particular embodiments, the user interface manager 914 generates and displays a user interface by way of a display screen composed of a plurality of graphical components, objects, and/or elements that allow a user to perform a function. For example, the user interface manager 914 receives user inputs from a user, such as a click/tap to select a field within an application. Additionally, in one or more embodiments, the user interface manager 914 presents a variety of types of information, including text, visual prompts, or other information for presentation in a user interface.

The data storage facility 916 maintains data for the switchboard management system 104. The data storage facility 916 (e.g., via one or more memory devices) maintains data of any type, size, or kind, as necessary to perform the functions of the switchboard management system 104. For example, the data storage facility 916 stores applications, digital mappings, etc.

Each of the components of the computing device 900 can include software, hardware, or both. For example, the components of the computing device 900 can include one or more instructions stored on a computer-readable storage medium and executable by processors of one or more computing devices, such as a client device or server device. When executed by the one or more processors, the computer-executable instructions of the switchboard management system 104 can cause the computing device(s) (e.g., the computing device 900) to perform the methods described herein. Alternatively, the components of the computing device 900 can include hardware, such as a special-purpose processing device to perform a certain function or group of functions. Alternatively, the components of the computing device 900 can include a combination of computer-executable instructions and hardware.

Furthermore, the components of the computing device 900 may, for example, be implemented as one or more operating systems, as one or more stand-alone applications, as one or more modules of an application, as one or more plug-ins, as one or more library functions or functions that may be called by other applications, and/or as a cloud-computing model. Thus, the components of the computing device 900 may be implemented as a stand-alone application, such as a desktop or mobile application. Furthermore, the components of the computing device 900 may be implemented as one or more web-based applications hosted on a remote server.

The components of the computing device 900 may also be implemented in a suite of mobile device applications or “apps.” To illustrate, the components of the computing device 900 may be implemented in a switchboard application, such as a mobile switchboard application or a desktop switchboard application.

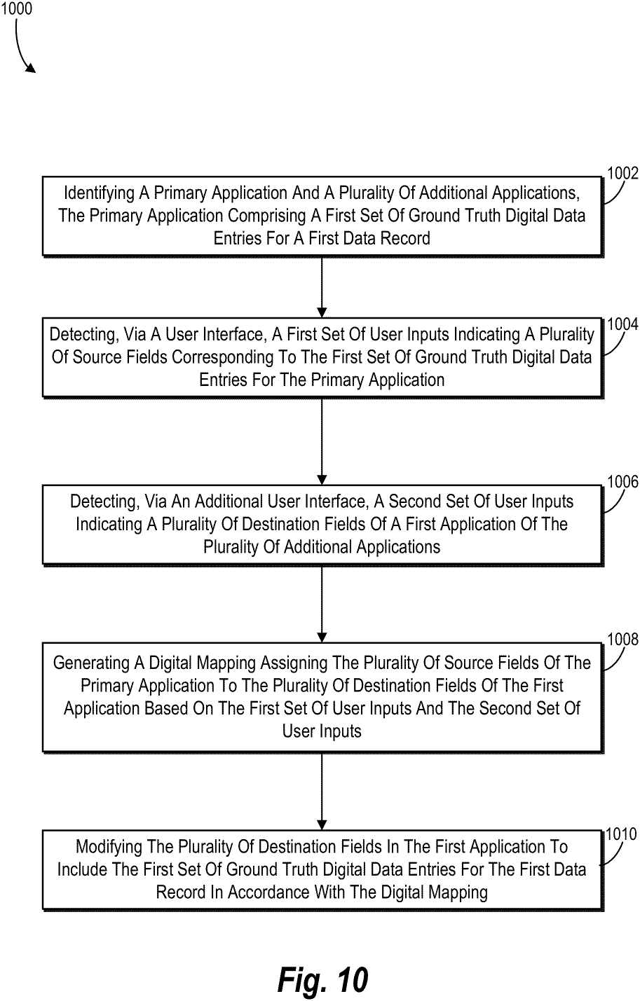

FIGS. 1-9, the corresponding text, and the examples provide several different systems, methods, techniques, components, and/or devices of the switchboard management system 104 in accordance with one or more embodiments. In addition to the above description, one or more embodiments can also be described in terms of flowcharts including acts for accomplishing a particular result. For example, FIG. 10 illustrates a flowchart of a series of acts 1000 for modifying a plurality of destination fields in accordance with one or more embodiments. The switchboard management system 104 may perform one or more acts of the series of acts 1000 in addition to or alternatively to one or more acts described in conjunction with other figures. While FIG. 10 illustrates acts according to one embodiment, alternative embodiments may omit, add to, reorder, and/or modify any of the acts shown in FIG. 10. The acts of FIG. 10 can be performed as part of a method. Alternatively, a non-transitory computer-readable medium can comprise instructions that, when executed by one or more processors, cause a computing device to perform the acts of FIG. 10. In some embodiments, a system can perform the acts of FIG. 10.

As shown, the series of acts 1000 includes an act 1002 of identifying a primary application and a plurality of additional applications, the primary application comprising a first set of ground truth digital data entries for a first data record.

The series of acts 1000 also includes an act 1004 of detecting, via a user interface, a first set of user inputs indicating a plurality of source fields corresponding to the first set of ground truth digital data entries for the primary application.

In addition, the series of acts 1000 includes an act 1006 of detecting, via an additional user interface, a second set of user inputs indicating a plurality of destination fields of a first application of the plurality of additional applications.

The series of acts 1000 further includes an act 1008 of generating a digital mapping assigning the plurality of source fields of the primary application to the plurality of destination fields of the first application based on the first set of user inputs and the second set of user inputs. In some embodiments, generating the digital mapping comprises generating mapping pairs for the plurality of source fields and the plurality of destination fields. Additionally or alternatively, in certain implementations, generating the digital mapping comprises utilizing a machine-learning model.

The series of acts 1000 also includes an act 1010 of modifying the plurality of destination fields in the first application to include the first set of ground truth digital data entries for the first data record in accordance with the digital mapping.

It is understood that the outlined acts in the series of acts 1000 are only provided as examples, and some of the acts may be optional, combined into fewer acts, or expanded into additional acts without detracting from the essence of the disclosed embodiments. Additionally, the acts described herein may be repeated or performed in parallel with one another or in parallel with different instances of the same or similar acts. As an example of an additional act not shown in FIG. 10, act(s) in the series of acts 1000 may include an act of: upon accessing the first data record via the primary application, populating a digital clipboard comprising the first set of ground truth digital data entries for the first data record; and utilizing the digital clipboard to transfer the first set of ground truth digital data entries to the plurality of destination fields in accordance with the digital mapping.

As another example of an additional act not shown in FIG. 10, act(s) in the series of acts 1000 may include an act of: in response to accessing a second data record via the primary application, clearing the digital clipboard; repopulating the digital clipboard with a second set of ground truth digital data entries for the second data record; and modifying the plurality of destination fields in the first application by utilizing the digital clipboard to transfer the second set of ground truth digital data entries in accordance with the digital mapping.

In yet another example of an additional act not shown in FIG. 10, act(s) in the series of acts 1000 may include an act of: detecting, via the user interface, a third set of user inputs indicating an additional plurality of source fields of the primary application corresponding to an additional set of ground truth digital data entries for a second data record; and detecting, via another user interface, a fourth set of user inputs indicating an additional plurality of destination fields of a second application of the plurality of additional applications.

Still further, in another example of an additional act not shown in FIG. 10, act(s) in the series of acts 1000 may include an act of: generating an additional digital mapping assigning the additional plurality of source fields of the primary application to the additional plurality of destination fields of the second application based on the third set of user inputs and the fourth set of user inputs; and modifying the additional plurality of destination fields of the second application to include the additional set of ground truth digital data entries in accordance with the additional digital mapping.

Additionally, in another example of an additional act not shown in FIG. 10, act(s) in the series of acts 1000 may include an act of: providing, for display within a first user interface, a first set of prompts for indicating the plurality of source fields; and providing, for display within a second user interface, a second set of prompts for indicating the plurality of destination fields.

As another example of an additional act not shown in FIG. 10, act(s) in the series of acts 1000 may include an act of: identifying user interaction to close or navigate away from the first data record within the primary application; and in response to identifying the user interaction, clearing a digital clipboard comprising the first set of ground truth digital data entries and clear the plurality of destination fields in the first application.

In yet another example of an additional act not shown in FIG. 10, act(s) in the series of acts 1000 may include an act of: determining the digital mapping comprises a mapping anomaly by comparing the predicted digital mapping to the digital mapping; and in response to determining the digital mapping comprises the mapping anomaly, providing a digital notification.

Embodiments of the present disclosure may comprise or utilize a special purpose or general-purpose computer including computer hardware, such as, for example, one or more processors and system memory, as discussed in greater detail below. Embodiments within the scope of the present disclosure also include physical and other computer-readable media for carrying or storing computer-executable instructions and/or data structures. In particular, one or more of the processes described herein may be implemented at least in part as instructions embodied in a non-transitory computer-readable medium and executable by one or more computing devices (e.g., any of the media content access devices described herein). In general, a processor (e.g., a microprocessor) receives instructions, from a non-transitory computer-readable medium, (e.g., memory), and executes those instructions, thereby performing one or more processes, including one or more of the processes described herein.

Computer-readable media can be any available media that can be accessed by a general purpose or special purpose computer system. Computer-readable media that store computer-executable instructions are non-transitory computer-readable storage media (devices). Computer-readable media that carry computer-executable instructions are transmission media. Thus, by way of example, and not limitation, embodiments of the disclosure can comprise at least two distinctly different kinds of computer-readable media: non-transitory computer-readable storage media (devices) and transmission media.

Non-transitory computer-readable storage media (devices) includes RAM, ROM, EEPROM, CD-ROM, solid state drives (“SSDs”) (e.g., based on RAM), Flash memory, phase-change memory (“PCM”), other types of memory, other optical disk storage, magnetic disk storage or other magnetic storage devices, or any other medium which can be used to store desired program code means in the form of computer-executable instructions or data structures and which can be accessed by a general purpose or special purpose computer.

A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmissions media can include a network and/or data links which can be used to carry desired program code means in the form of computer-executable instructions or data structures and which can be accessed by a general purpose or special purpose computer. Combinations of the above should also be included within the scope of computer-readable media.

Further, upon reaching various computer system components, program code means in the form of computer-executable instructions or data structures can be transferred automatically from transmission media to non-transitory computer-readable storage media (devices) (or vice versa). For example, computer-executable instructions or data structures received over a network or data link can be buffered in RAM within a network interface module (e.g., a “NIC”), and then eventually transferred to computer system RAM and/or to less volatile computer storage media (devices) at a computer system. Thus, it should be understood that non-transitory computer-readable storage media (devices) can be included in computer system components that also (or even primarily) utilize transmission media.

Computer-executable instructions comprise, for example, instructions and data which, when executed by a processor, cause a general-purpose computer, special purpose computer, or special purpose processing device to perform a certain function or group of functions. In some embodiments, computer-executable instructions are executed by a general-purpose computer to turn the general-purpose computer into a special purpose computer implementing elements of the disclosure. The computer-executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Those skilled in the art will appreciate that the disclosure may be practiced in network computing environments with many types of computer system configurations, including, personal computers, desktop computers, laptop computers, message processors, hand-held devices, multiprocessor systems, microprocessor-based or programmable consumer electronics, network PCs, minicomputers, mainframe computers, mobile telephones, PDAs, tablets, pagers, routers, switches, and the like. The disclosure may also be practiced in distributed system environments where local and remote computer systems, which are linked (either by hardwired data links, wireless data links, or by a combination of hardwired and wireless data links) through a network, both perform tasks. In a distributed system environment, program modules may be located in both local and remote memory storage devices.

Embodiments of the present disclosure can also be implemented in cloud computing environments. As used herein, the term “cloud computing” refers to a model for enabling on-demand network access to a shared pool of configurable computing resources. For example, cloud computing can be employed in the marketplace to offer ubiquitous and convenient on-demand access to the shared pool of configurable computing resources. The shared pool of configurable computing resources can be rapidly provisioned via virtualization and released with low management effort or service provider interaction, and then scaled accordingly.

A cloud-computing model can be composed of various characteristics such as, for example, on-demand self-service, broad network access, resource pooling, rapid elasticity, measured service, and so forth. A cloud-computing model can also expose various service models, such as, for example, Software as a Service (“SaaS”), Platform as a Service (“PaaS”), and Infrastructure as a Service (“IaaS”). A cloud-computing model can also be deployed using different deployment models such as private cloud, community cloud, public cloud, hybrid cloud, and so forth. In addition, as used herein, the term “cloud-computing environment” refers to an environment in which cloud computing is employed.

FIG. 11 illustrates a block diagram of an example computing device 1100 that may be configured to perform one or more of the processes described above. One will appreciate that one or more computing devices, such as the computing device 1100 may represent the computing devices described above (e.g., the server(s) 102, the client device 106, the administrator device 110, the third-party server(s) 114, the computing device 400, and/or the computing device 900). In one or more embodiments, the computing device 1100 may be a mobile device (e.g., a mobile telephone, a smartphone, a PDA, a tablet, a laptop, a camera, a tracker, a watch, a wearable device, etc.). In some embodiments, the computing device 1100 may be a non-mobile device (e.g., a desktop computer or another type of client device). Further, the computing device 1100 may be a server device that includes cloud-based processing and storage capabilities.

As shown in FIG. 11, the computing device 1100 can include one or more processor(s) 1102, memory 1104, a storage device 1106, input/output interfaces 1108 (or “I/O interfaces 1108”), and a communication interface 1110, which may be communicatively coupled by way of a communication infrastructure (e.g., bus 1112). While the computing device 1100 is shown in FIG. 11, the components illustrated in FIG. 11 are not intended to be limiting. Additional or alternative components may be used in other embodiments. Furthermore, in certain embodiments, the computing device 1100 includes fewer components than those shown in FIG. 11. Components of the computing device 1100 shown in FIG. 11 will now be described in additional detail.

In particular embodiments, the processor(s) 1102 includes hardware for executing instructions, such as those making up a computer program. As an example, and not by way of limitation, to execute instructions, the processor(s) 1102 may retrieve (or fetch) the instructions from an internal register, an internal cache, memory 1104, or a storage device 1106 and decode and execute them.

The computing device 1100 includes memory 1104, which is coupled to the processor(s) 1102. The memory 1104 may be used for storing data, metadata, and programs for execution by the processor(s). The memory 1104 may include one or more of volatile and nonvolatile memories, such as Random-Access Memory (“RAM”), Read-Only Memory (“ROM”), a solid-state disk (“SSD”), Flash, Phase Change Memory (“PCM”), or other types of data storage. The memory 1104 may be internal or distributed memory.

The computing device 1100 includes a storage device 1106 includes storage for storing data or instructions. As an example, and not by way of limitation, the storage device 1106 can include a non-transitory storage medium described above. The storage device 1106 may include a hard disk drive (HDD), flash memory, a Universal Serial Bus (USB) drive or a combination these or other storage devices.

As shown, the computing device 1100 includes one or more I/O interfaces 1108, which are provided to allow a user to provide input to (such as user strokes), receive output from, and otherwise transfer data to and from the computing device 1100. These I/O interfaces 1108 may include a mouse, keypad or a keyboard, a touch screen, camera, optical scanner, network interface, modem, other known I/O devices or a combination of such I/O interfaces 1108. The touch screen may be activated with a stylus or a finger.

The I/O interfaces 1108 may include one or more devices for presenting output to a user, including, but not limited to, a graphics engine, a display (e.g., a display screen), one or more output drivers (e.g., display drivers), one or more audio speakers, and one or more audio drivers. In certain embodiments, I/O interfaces 1108 are configured to provide graphical data to a display for presentation to a user. The graphical data may be representative of one or more graphical user interfaces and/or any other graphical content as may serve a particular implementation.

The computing device 1100 can further include a communication interface 1110. The communication interface 1110 can include hardware, software, or both. The communication interface 1110 provides one or more interfaces for communication (such as, for example, packet-based communication) between the computing device and one or more other computing devices or one or more networks. As an example, and not by way of limitation, communication interface 1110 may include a network interface controller (NIC) or network adapter for communicating with an Ethernet or other wire-based network or a wireless NIC (WNIC) or wireless adapter for communicating with a wireless network, such as a WI-FI. Moreover, in some embodiments, the communication interface 1110 comprises hardware and/or software for encrypting and decrypting digital communications, whether stored on the computing device 1100 and/or communicated to other computing devices. The computing device 1100 can further include a bus 1112. The bus 1112 can include hardware, software, or both that connects components of the computing device 1100 to each other.

In the foregoing specification, the invention has been described with reference to specific example embodiments thereof. Various embodiments and aspects of the invention(s) are described with reference to details discussed herein, and the accompanying drawings illustrate the various embodiments. The description above and drawings are illustrative of the invention and are not to be construed as limiting the invention. Numerous specific details are described to provide a thorough understanding of various embodiments of the present invention.

The present invention may be embodied in other specific forms without departing from its spirit or essential characteristics. The described embodiments are to be considered in all respects only as illustrative and not restrictive. For example, the methods described herein may be performed with less or more steps/acts or the steps/acts may be performed in differing orders. Additionally, the steps/acts described herein may be repeated or performed in parallel to one another or in parallel to different instances of the same or similar steps/acts. The scope of the invention is, therefore, indicated by the appended claims rather than by the foregoing description. All changes that come within the meaning and range of equivalency of the claims are to be embraced within their scope.

What is claimed is:

1. A non-transitory computer-readable storage medium comprising instructions that, when executed by at least one processor, cause a computing device to:
identify a primary application and a plurality of additional applications, the primary application comprising a first set of ground truth digital data entries for a first data record;
detect, via a user interface, a first set of user inputs indicating a plurality of source fields corresponding to the first set of ground truth digital data entries for the primary application;
detect, via an additional user interface, a second set of user inputs indicating a plurality of destination fields of a first application of the plurality of additional applications;
generate a digital mapping assigning the plurality of source fields of the primary application to the plurality of destination fields of the first application based on the first set of user inputs and the second set of user inputs; and
modify the plurality of destination fields in the first application to include the first set of ground truth digital data entries for the first data record in accordance with the digital mapping.

2. The non-transitory computer-readable storage medium of claim 1, further comprising instructions that, when executed by the at least one processor, cause the computing device to:
upon accessing the first data record via the primary application, populate a digital clipboard comprising the first set of ground truth digital data entries for the first data record; and
utilize the digital clipboard to transfer the first set of ground truth digital data entries to the plurality of destination fields in accordance with the digital mapping.

3. The non-transitory computer-readable storage medium of claim 2, further comprising instructions that, when executed by the at least one processor, cause the computing device to:
in response to accessing a second data record via the primary application, clear the digital clipboard;
repopulate the digital clipboard with a second set of ground truth digital data entries for the second data record; and
modify the plurality of destination fields in the first application by utilizing the digital clipboard to transfer the second set of ground truth digital data entries in accordance with the digital mapping.

4. The non-transitory computer-readable storage medium of claim 1, further comprising instructions that, when executed by the at least one processor, cause the computing device to:
detect, via the user interface, a third set of user inputs indicating an additional plurality of source fields of the primary application corresponding to an additional set of ground truth digital data entries for a second data record; and
detect, via another user interface, a fourth set of user inputs indicating an additional plurality of destination fields of a second application of the plurality of additional applications.

5. The non-transitory computer-readable storage medium of claim 4, further comprising instructions that, when executed by the at least one processor, cause the computing device to:
generate an additional digital mapping assigning the additional plurality of source fields of the primary application to the additional plurality of destination fields of the second application based on the third set of user inputs and the fourth set of user inputs; and
modify the additional plurality of destination fields of the second application to include the additional set of ground truth digital data entries in accordance with the additional digital mapping.

6. The non-transitory computer-readable storage medium of claim 1, further comprising instructions that, when executed by the at least one processor, cause the computing device to generate the digital mapping by generating mapping pairs for the plurality of source fields and the plurality of destination fields.

7. The non-transitory computer-readable storage medium of claim 1, further comprising instructions that, when executed by the at least one processor, cause the computing device to:
provide, for display within a first user interface, a first set of prompts for indicating the plurality of source fields; and
provide, for display within a second user interface, a second set of prompts for indicating the plurality of destination fields.

8. The non-transitory computer-readable storage medium of claim 1, further comprising instructions that, when executed by the at least one processor, cause the computing device to:
identify a user interaction to close or navigate away from the first data record within the primary application; and
in response to identifying the user interaction, clear a digital clipboard comprising the first set of ground truth digital data entries and clear the plurality of destination fields in the first application.

9. The non-transitory computer-readable storage medium of claim 1, further comprising instructions that, when executed by the at least one processor, cause the computing device to generate a predicted digital mapping utilizing a machine-learning model.

10. The non-transitory computer-readable storage medium of claim 9, further comprising instructions that, when executed by the at least one processor, cause the computing device to:
determine the digital mapping comprises a mapping anomaly by comparing the predicted digital mapping to the digital mapping; and
in response to determining the digital mapping comprises the mapping anomaly, provide a digital notification.

11. A computer-implemented method comprising:
identifying a primary application and a plurality of additional applications, the primary application comprising a first set of ground truth digital data entries for a first data record;
detecting, via a user interface, a first set of user inputs indicating a plurality of source fields corresponding to the first set of ground truth digital data entries for the primary application;
detecting, via an additional user interface, a second set of user inputs indicating a plurality of destination fields of a first application of the plurality of additional applications;
generating a digital mapping assigning the plurality of source fields of the primary application to the plurality of destination fields of the first application based on the first set of user inputs and the second set of user inputs; and
modifying the plurality of destination fields in the first application to include the first set of ground truth digital data entries for the first data record in accordance with the digital mapping.

12. The computer-implemented method of claim 11, further comprising:
upon accessing the first data record via the primary application, populating a digital clipboard comprising the first set of ground truth digital data entries for the first data record; and
utilize the digital clipboard to transfer the first set of ground truth digital data entries to the plurality of destination fields in accordance with the digital mapping.

13. The computer-implemented method of claim 12, further comprising:
in response to accessing a second data record via the primary application, clearing the digital clipboard;
repopulating the digital clipboard with a second set of ground truth digital data entries for the second data record; and
modifying the plurality of destination fields in the first application by utilizing the digital clipboard to transfer the second set of ground truth digital data entries in accordance with the digital mapping.

14. The computer-implemented method of claim 11, further comprising:
detecting, via the user interface, a third set of user inputs indicating an additional plurality of source fields of the primary application corresponding to an additional set of ground truth digital data entries for a second data record; and
detecting, via another user interface, a fourth set of user inputs indicating an additional plurality of destination fields of a second application of the plurality of additional applications.

15. The computer-implemented method of claim 14, further comprising:
generating an additional digital mapping assigning the additional plurality of source fields of the primary application to the additional plurality of destination fields of the second application based on the third set of user inputs and the fourth set of user inputs; and
modifying the additional plurality of destination fields of the second application to include the additional set of ground truth digital data entries in accordance with the additional digital mapping.

16. A system comprising:
at least one processor; and
a non-transitory computer readable storage medium comprising instructions that, when executed by the at least one processor, cause the system to:
identify a primary application and a plurality of additional applications, the primary application comprising a first set of ground truth digital data entries for a first data record;
detect, via a user interface, a first set of user inputs indicating a plurality of source fields corresponding to the first set of ground truth digital data entries for the primary application;
detect, via an additional user interface, a second set of user inputs indicating a plurality of destination fields of a first application of the plurality of additional applications;
generate a digital mapping assigning the plurality of source fields of the primary application to the plurality of destination fields of the first application based on the first set of user inputs and the second set of user inputs; and
modify the plurality of destination fields in the first application to include the first set of ground truth digital data entries for the first data record in accordance with the digital mapping.

17. The system of claim 16, further comprising instructions that, when executed by the at least one processor, cause the system to:
upon accessing the first data record via the primary application, populate a digital clipboard comprising the first set of ground truth digital data entries for the first data record; and
utilize the digital clipboard to transfer the first set of ground truth digital data entries to the plurality of destination fields in accordance with the digital mapping.

18. The system of claim 17, further comprising instructions that, when executed by the at least one processor, cause the system to:
in response to accessing a second data record via the primary application, clear the digital clipboard;
repopulate the digital clipboard with a second set of ground truth digital data entries for the second data record; and
modify the plurality of destination fields in the first application by utilizing the digital clipboard to transfer the second set of ground truth digital data entries in accordance with the digital mapping.

19. The system of claim 16, further comprising instructions that, when executed by the at least one processor, cause the system to:
provide, for display within a first user interface, a first set of prompts for indicating the plurality of source fields; and
provide, for display within a second user interface, a second set of prompts for indicating the plurality of destination fields.

20. The system of claim 16, further comprising instructions that, when executed by the at least one processor, cause the system to:
identify a user interaction to close or navigate away from the first data record within the primary application; and
in response to identifying the user interaction, clear a digital clipboard comprising the first set of ground truth digital data entries and clear the plurality of destination fields in the first application.
